(19) United States
(12) Patent Application Publication
(10) Pub. No.: US 2016/0369076 A1
(43) Pub. Date: Dec. 22, 2016

(54) SUPER HYDROPHOBIC MULTISCALE POROUS POLYMER FILMS

(71) Applicant: Board of Trustees of Michigan State University, East Lansing, MI (US)

(72) Inventors: Ilsoon LEE, Okemos, MI (US); Jing YU, East Lansing, MI (US); Oishi SANYAL, East Lansing, MI (US)

(21) Appl. No.: 15/157,526

(22) Filed: May 18, 2016

Related U.S. Application Data
(63) Continuation-in-part of application No. 14/937,955, filed on Nov. 11, 2015.
(60) Provisional application No. 62/254,371, filed on Nov. 12, 2015.
(60) Provisional application No. 62/180,982, filed on Jun. 17, 2015.
(60) Provisional application No. 62/080,296, filed on Nov. 15, 2014.
(60) Provisional application No. 62/080,010, filed on Nov. 14, 2014.

(51) Int. Cl. C08J 9/00; A61K 9/70; C09D 179/02; A61K 47/34; C09D 133/02; C09D 5/16; A61K 47/32

(52) U.S. Cl. CPC C08J 9/0061; C09D 5/1681; C09D 5/1693; C09D 5/1662; A61K 9/7007; A61K 47/32; A61K 47/34; C09D 133/02; C09D 179/02; C08J 2333/02; C08J 2379/02; C08J 2205/042; C08J 2205/044

(57) ABSTRACT

Porous polyelectrolyte multilayer (PEM) films with pore size control ranging from nano- to micro-scale are made hydrophobic by coating with fluorine compounds. A layer-by-layer (LbL) technique is used to fabricate PEMs, and the built up PEMs are subject to subsequent porous treatment under acidic or basic conditions. Besides shortening the processing time, polyelectrolytes with high molecular weight are used for the first time. Multi-scale porous structures are provided with either micro-sized porous structure on top of nano-sized porous structure or the other way around.

### CROSS-REFERENCE TO RELATED APPLICATIONS

This application claims the benefit of U.S. Provisional Application No. 62/254,371 filed on Nov. 12, 2015, and is a continuation-in-part of U.S. patent application Ser. No. 14/937,955 filed on Nov. 11, 2015, which claims the benefit of U.S. Provisional Applications No. 62/180,982 filed on Jun. 17, 2015; 62/080,296 filed on Nov. 15, 2014; and 62/080,010 filed on Nov. 14, 2014. The entire disclosures of the above applications are incorporated herein by reference.

### GOVERNMENT SUPPORT

This invention was made with Government support under W912HQ-12-C-0020 awarded by the Department of Defense, Strategic Environmental Research and Development Program (SERDP). The U. S. Government has certain rights in this invention.

### BACKGROUND

This section provides background information related to the present disclosure which is not necessarily prior art.

Layer-by-layer (LbL) assembled PEMs have been considered as a versatile platform for surface modification. In 1990s, Gero Decher pioneered the LbL technique to build multilayers by dipping a positive-charged substrate into polyanion and polycation solutions alternately, resulting in PEMs with precise structure control in nanometer scale. (Decher G. Science, 1997, 277(5330): 1232-1237.) For conventional LbL process, the dipping time for polyanion and polycation solutions is around 10 minutes or more. Sufficient washing steps are also required. And, to achieve a proper thickness, 10 bilayers or more are always needed. Thus, slow processing becomes one of the major issues for the industrializing PEM products. The short-time LbL technique originally pioneered by Grunlan et al., (Hagen D A, Foster B, Stevens B, et al. ACS Macro Letters, 2014, 3(7): 663-666.) is an effective tool for fabricating porous PEM structures.

The conventional LbL process is extremely time consuming and it takes several hours to fabricate a PEM film of desired thickness. Hence, although the process has been developed more than a decade ago, and extensive research, both in terms of fundamentals as well as applications have been carried out, this LbL process has not seen industrial acceptance.

Multi-scale porous structures have been successfully built up either with a micro-sized porous structure on top of a nano-sized porous structure or the other way around. According to the previous studies about porous PEM films, either only one porous structure was developed in one sample (Hiller J A, et al., Nature Materials, 2002, 1(1): 59-63; Berg M C, et al., Biomacromolecules, 2006, 7(1): 357-364; Cho C, Zacharia N S. Langmuir, 2011, 28(1): 841-848), or with micro- and nano-structures on top of the surface randomly (Fu J, Ji J, Shen L, et al. Langmuir, 2008, 25(2): 672-675.).

### SUMMARY

This section provides a general summary of the disclosure, and is not a comprehensive disclosure of its full scope or all of its features.

Porous polyelectrolyte multilayer (PEM) films have been created with precise pore size control ranging from nano- to micro-scale. Layer-by-layer (LbL) technique has been applied for fabricating PEMs, and the porous treatment has been carried out under acidic condition.

The primary purpose of our work aims at reducing the processing time of the process to fabricate porous PEM films without compromising on the quality of the prepared films. This will enable the tremendously versatile LbL coating process to be economically fabricated. Besides shortening the processing time, we also tried polyelectrolytes with high molecular weight. This enables a broader control of pore size. Multi-scale porous structures have been first developed in this work, with either micro-sized porous structure on top of nano-sized porous structure or the reverse.

Porous polyelectrolyte multilayer (PEM) films have been fabricated via fast layer-by-layer (LbL) technique, followed by acidic treatment with pH varying from 1.8 to 2.4. In our approach, the dipping time has been shortened significantly. The dipping time can be as short as 10 seconds or can be extended to about 15 minutes. The film thickness can be tuned by manipulating dipping time, molecular weight, number of bilayers, etc.

In this work, we use Poly(acrylic acid) (PAA) as the polyanion, and Poly(allylamine hydrochloride)(PAH) as polycation. In order to achieve a broader control of pore size, PAA with high molecular weight (M_{w}=225,000 g/mol) has been tried. This high molecular weight PAA can form special microfibrous structure on the surface via LbL assembly with dipping time longer than 5 minutes. However, with 10 second dipping, the surface is flat and smooth, and after porous treatment with pH of 2.0, pore size of 20-50 nm can be obtained, which is much smaller than what has been reported in literatures. (Cho C, Zacharia N S. Langmuir, 2011, 28(1): 841-848. Berg M C, et al., Biomacromolecules, 2006, 7(1): 357-364.)

In this invention, we can control the micro-sized and nano-sized porous regions. To fabricate multi-scale porous structure, we first make the bottom porous structure via LbL assembly followed by acid treatment and crosslink the structure. Then the top porous structure can be further built up through the same way. If the bottom porous structure is with a nano-sized structure, the top porous structure can be built up with no need to consider the molecular weight of polyelectrolytes. However, if the bottom has micro-sized porous structure, higher molecular weight of polyelectrolytes is required for the top porous structure since the polymer chain needs to be large enough to avoid filling into bottom pores.

This invention has potential for various applications, such as 1) membranes, 2) drug delivery, and 3) super hydrophobic coatings (i.e., self-cleaning). For drug delivery, the porous structure can be considered as a drug reservoir. This invention allows the design of certain porous structures to fulfill the release requirements, such as initial burst release, sustained release, or a combination of both. The release kinetics can be precisely controlled by tuning the porous structure. For membrane applications, these porous PEM structures can also be used to replace the porous polysulfone and polyamide layers of reverse osmosis (RO) membranes. In addition, micro- and nano-structured surface can be achieved with our approach, which can be further modified by fluorinated silane molecules to obtain super hydrophobic (self-cleaning) surfaces.

### DRAWINGS

The drawings described herein are for illustrative purposes only of selected embodiments and not all possible implementations, and are not intended to limit the scope of the present disclosure.

Corresponding reference numerals indicate corresponding parts throughout the several views of the drawings.

FIG. 1 illustrates polyelectrolytes used.

FIG. 2 details the LbL process.

FIG. 3 is a graph of thickness of PEM film formed as a function of dipping time.

FIGS. 4-6 show micrographs of PEM films.

FIG. 7 is a graph of roughness as a function of dipping time.

FIGS. 8-12 show micrographs of PEM films.

FIGS. 13 and 14 show graphs of film thickness as a function of dipping time, molecular weight of polyelectrolyte, and pH of porous treatment.

FIGS. 15-17 show micrographs of PEM thin films.

FIG. 18 shows XPS results from treated PEM films.

FIG. 19 is a photo of a free-standing film prepared according to the described methods.

FIGS. 20-23 show micrographs illustrating design of multiscale porous structures.

FIG. 24 shows the effect of dipping time on (a) the thickness of PAH_{L}/PAA_{L} thin films before and after the post treatment and (b) the relative expansion of thickness and average surface pore size.

In FIG. 25, FIGS. 25A and 25B, 25C and 25D, 25E and 25F, 25G and 25H, and 25I and 25J are the top-view and cross-sectional SEM images for porous (PAH_{L}/PAA_{L})_{20.5} films with dipping time of 10 s, 1 min, 5 min, 10 min and 15 min, respectively. The arrow in each cross-sectional image indicates the interface between the glass substrate and the deposited film.

FIG. 26 shows (a) thickness of thin films before and after the post treatment (FIG. 26A) and (b) the relative expansion of thickness and average surface pore size for (PAH_{L}/PAA_{L})_{20.5}, (PAH_{H}/PAA_{L})_{20.5}, (PAH_{L}/PAA_{H})_{20.5}, and (PAH_{H}/PAA_{H})_{20.5} (FIG. 26B). All the films were fabricated using dipping time of 10 s.

In FIG. 27, FIGS. 27A and 27B, 27C and 27D, 27E and 27F, and 27G and 27H are the top-view and cross-sectional SEM images for porous (PAH_{L}/PAA_{L})_{20.5}, (PAH_{H}/PAA_{L})_{20.5}, (PAH_{L}/PAA_{H})_{20.5}, and (PAH_{H}/PAA_{H})_{20.5} films, respectively. The arrow in each cross-sectional image indicates the interface between the glass substrate and the deposited film. All the films were fabricated using dipping time of 10 s.

FIG. 28 shows SEM cross-sectional (FIG. 28A and FIG. 28B) and top view (FIG. 28C) images of multi-scale porous thin films with nano-sized porous film as the bottom and micro-sized porous film as the top. The arrow in FIG. 28A indicates the interface between the glass substrate and the deposited film. FIG. 28B is an enlarged image of the square area in FIG. 28A.

FIG. 29 shows SEM cross-sectional (FIG. 29A and FIG. 29B) and top view (FIG. 29C) images of multi-scale porous thin films with micro-sized porous structure as the bottom and nano-sized porous structure as the top. The arrow in FIG. 29A indicates the interface between the glass substrate and the deposited film. FIG. 29B is an enlarged image of the square area in FIG. 29A.

FIGS. 30A, 30B, 30C, 30D, and 30E show SEM images of the porous (PAH_{H}/PAA_{L})_{20.5} surfaces with dipping time of 10 s, 1 min, 5 min, 10 min and 15 min, respectively. (The porous induction was done at pH of 2.0.)

FIG. 31 shows the values of contact angle for porous (PAH_{L}/PAA_{L})_{20.5} and (PAH_{H}/PAA_{L})_{20.5} with different dipping time.

FIG. 32 shows super hydrophilic and super hydrophobic surfaces achieved before and after CVD for porous PAH_{H}/PAA_{L} thin film with dipping time of 1 min and acidic treatment at pH=2.0.

FIGS. 33A, 33B, 33C, 33D, and 33E show images of the (PAH_{L}/PAA_{L})_{20.5} surfaces with dipping time of 10 s, 1 min, 5 min, 10 min and 15 min, respectively.

FIGS. 34A, 34B, 34C, 34D, and 34E show images of the (PAH_{L}/PAA_{H})_{20.5} surfaces with dipping time of 10 s, 1 min, 5 min, 10 min and 15 min, respectively.

FIG. 35 shows the values of (FIG. 35A) roughness and (FIG. 35B) contact angle for (PAH_{L}/PAA_{L})_{20.5} and (PAH_{L}/PAA_{H})_{20.5} with different dipping time.

FIGS. 36A, 36B, 36C, 36D, and 36E show SEM images of the porous (PAH_{L}/PAA_{L})_{20.5} surfaces with dipping time of 10 s, 1 min, 5 min, 10 min and 15 min, respectively. (The porous induction was done at pH of 2.0.)

FIGS. 37A, 37B, 37C, 37D, and 37E show SEM images of the porous (PAH_{H}/PAA_{L})_{20.5} surfaces with dipping time of 10 s, 1 min, 5 min, 10 min and 15 min, respectively. (The porous induction was done at pH of 2.0.)

FIG. 38 shows the values of (FIG. 38A) roughness and (FIG. 38B) contact angle for (PAH_{L}/PAA_{L})_{20.5} and (PAH_{L}/PAA_{H})_{20.5} with different dipping time. (The porous induction was done at pH of 2.0.)

FIGS. 39A, 39B, 39C, and 39D show SEM images of the porous (PAH_{H}/PAA_{L})_{20.5} surfaces (dipping time of 1 min) with porous induction at pH 1.8, 2.0, 2.2 and 2.4, respectively.

### DETAILED DESCRIPTION

Example embodiments will now be described more fully with reference to the accompanying drawings.

### Layer by Layer (LbL) Assembly of Polyelectrolyte Multilayer Thin Films

Composites containing a PEM film on a substrate are prepared layer-by-layer by sequentially applying layers of polycation and polyanion on a substrate, such as a silicon, glass, plastic slide, or—as disclosed below—a non-woven web or non-woven fabric. A wide range of negatively charged and positively charged polymers is suitable for making the layered materials. Suitable polymers are water soluble and sufficiently charged (by virtue of the chemical structure and/or the pH state of the solutions) to form a stable electrostatic assembly of electrically charged polymers. Sulfonated polymers such as sulfonated polystyrene are commonly used as the negatively charged polyelectrolyte. Quaternary nitrogen-containing polymers such as poly (diallyldimethylammonium chloride) (PDAC) are commonly used as the positively charged electrolyte.

Polyelectrolytes include positively and negatively charged polymers, and are also divided among “strong” and “weak” polyelectrolytes depending on whether the charged groups do or do not maintain their charge over a wide pH range. For example, a sulfonated polymer is considered a strong polyelectrolyte because it is negatively charged over a wide pH range; an acrylic acid polymer is considered a weak polyelectrolyte because it is protonated below a pH of about 4 but contains a negative charge at higher pH. Strong polyelectrolytes include sulfonated polystyrene (SPS) and poly (diallyldimethyl ammonium chloride) (PDAC). Weak polyelectrolytes include polyacrylics such as polyacrylic acid, as well as positively charged polyelectrolytes such as poly (allyl amine) and branched and linear polyethyleneimines as their respective ammonium salts.

In various embodiments, polyelectrolyte multilayers are prepared by applying a first charged polyelectrolyte to a substrate surface by electrostatic interaction. The nature of the first polyelectrolyte applied (polyanion or polycation) depends on the charge state of the substrate surface. Thereafter, additional layers of polyelectrolyte are deposited in alteration between positive and negative. If a substrate surface is hydrophobic and not capable of electrostatic interactions with a polyelectrolyte (an example is an un-plasma treated silicone surface), it is possible to apply a first polyelectrolyte that interacts with the hydrophobic surface by hydrophobic interactions, but that is capable of interacting with a subsequent polyelectrolyte layer. For example, layers of PDAC/SPS cannot be assembled on a hydrophobic (non-plasma treated) surface of PDMS. However by starting with one layer of PAH, at a pH of 7.5, SPS/PDAC can be assembled on PDMS, where PAH interacts with PDMS by hydrophobic interactions and SPS/PDAC can be built on the PAH by electrostatic interactions. This is further explained and illustrated in Park et al., Advanced Materials 16, 520-525 (2004), the disclosure of which provides background information and is hereby incorporated by reference.

Applying the polycation and polyanion and building up the alternating layers of polyelectrolyte on the substrate are accomplished with any suitable method. In a first method, the substrate or a substrate containing built-up layers is dipped or immersed in a solution of polycation or polyanion. After each application of polyelectrolyte, the substrate is removed and is preferably rinsed. Following the rinse step, the substrate is dipped or immersed again in a solution of the oppositely charged polyelectrolyte. Following a rinse step, the process is repeated as desired to build up a number of layers. This layer by layer assembly method is well known and is described for example in Decher, Science 277, 1232 (1997), the disclosure of which is helpful for background information and is hereby incorporated by reference.

In other embodiments, the polyelectrolytes are applied by 1) spin casting, 2) solution casting, or 3) spray assembly. After application of one layer, the applied layer is preferably rinsed before the next layer is applied. In this way, alternating layers of polycation and polyanion are applied to the surface until the desired number of bilayers is achieved.

Methods of assembling the PEMs are well known. The methods can be conveniently automated with robots. Polycation and polyanion is alternately applied layer-by-layer to a substrate. When the substrate surface is capable of electrostatic interactions with a positively charged material (that is, when it is negatively charged), a polycation is first applied to the substrate, preferably followed by a rinse step. The polycation is followed with application of a polyanion. The procedure is repeated as desired until a number of layers are built up. A bilayer consists of a layer of polycation and a layer of polyanion. Thus for example, 10 bilayers contain 20 layers, while 10.5 bilayers contain 21 layers. With an integer number of bilayers, the top surface of the PEM has the same charge as the substrate. With a half bi-layer (e.g. 10.5 illustrated) the top surface of the PEM is oppositely charged to the substrate.

Multilayer films are abbreviated as (x/y)_{z} where x is the first polyion deposited, y is the second polyelectrolyte deposited and z is the number of bilayers. Half a bilayer means that x was the last polyelectrolyte deposited.

### Pores, Nanopores and Micropores

Pores in the PEM thin films described herein are classified as nanopores or micropores depending on their size. Nanopores are characterized by dimensions on the order of nanometers, and in any event less than 1 micron (which equals 1000 nm). Micropores where used indicates a pore with a dimension of 1 micron or greater. In one embodiment described herein, a film contains nanopores having a dimension of 20 nm to 50 nm. For pores that are nearly circular, such as many of the nanopores illustrated in the Figures, the word diameter can be used interchangeably for the size of the pore. But the use of “diameter” here or in the claims is not to be taken as an indication that the description is limited to round or perfectly round pores. Rather, it is a short hand way to describe the minimum dimension of a pore; if that minimum dimension is less than a micron, it is a nanopore.

### Dipping Time

An important parameter in LbL assembly is the dipping time, or the time for which the growing film is exposed to solutions of polyanion or polycation. As detailed herein, dipping varies from 1 second, to 10 seconds, up to 15 minutes. Dipping is at room temperature unless otherwise stated. It has been discovered that the morphology the PEM film can be designed and altered by selecting suitable values for dipping times and the nature and molecular weight of the polyelectrolytes.

### Porous PEM Films and Methods for Making

In one embodiment, a polyelectrolyte multilayer (PEM) thin film is made having pores in the film, and wherein at least some of the pores have a diameter in a range of 20-50 nm. In various embodiments, the film contains alternating layers of polycation and polyacrylic acid, with the polyacrylic acid preferably having a higher weight average (molecular) than conventional polyacrylics used in the LbL technique. In a non-limiting embodiment, the weight average molecular weight of polyacrylic acid is over 100,000 g/mol. Methods of making the PEM thin film are also provided.

In another embodiment, a PEM thin film has a hydrophobic surface characterized by a contact angle with water of greater than 150°, and in some embodiments, 160° or greater. In various embodiments, the film contains alternating layers of polycation and polyacrylic acid. In an exemplary embodiment, the weight average molecular weight of the polyacrylic acid is greater than 100,000, for example about 225,000 g/mol. In various embodiments, the thin film is porous treated to introduce nanoporous features into a microfibrous morphology of the thin film. Optionally, the surface of the PEM thin film comprises a fluorine compound that contributes to hydrophobicity or super hydrophobicity of the surface. In exemplary embodiments, the fluorine compound is bound to the surface by a covalent attachment of a silane functional group of the fluorine compound to a functional group on the surface of the thin film.

In another embodiment, a PEM thin film is made of built-up alternating layers of polycation and polyanion, wherein the polyanion comprises polyacrylic acid having a weight average molecular weight above that conventionally used for PEM thin films. In various embodiments, the polyacrylic acid has a weight average molecular weight greater than 50,000, greater than 60,000, greater than 70,000, greater than 80,000, or greater than 100,000 g/mol. In an exemplary embodiment, a polyacrylic acid with a weight average molecular weight of about 225,000 g/mol is used. Optionally the surface is covered or partially covered with a fluorine compound, as in the preceding paragraph.

In another embodiment, a method of making a PEM thin film involves layer by layer assembly of alternating polycation and polyanion. In the method, a first layer of polycation is applied to a negatively charged substrate, and alternating layers of polycation and polyanion are built up to make the thin film. The method involves alternately dipping the coated substrate in a solution containing polyanion and then in a solution containing polycation. The dipping time in each solution is 1 minute or less, in order to provide a smooth morphology that can be subsequently porous treated. As in some other embodiments, the polyanion comprises a polyacrylic acid that has weight average molecular weight higher than the weight of polyacrylic acid normally used in LbL procedures. In various embodiments, the molecular weight (weight average) of an acrylic polymer used in the method is above 100,000 g/mol.

In another embodiment, the LbL assembly method described in the preceding paragraph is used, but the dipping time in each solution (of polyanion and of polycation) is greater than 1 minute. The use of a polyacrylic acid having an above normal molecular weight (e.g. above 100,000 g/mol), together with the dipping time greater than 1 minute, leads to microfibrous morphology, which can be subsequently porous treated to provide microporous structure.

In the various methods of making PEM thin films by LbL assembly of polycation and polyanion wherein the polyanion has a high molecular weight (such as above 100,000 or about 225,000), the film is post treated and then exposed to a fluorine containing molecule in the gas phase to produce a super hydrophobic surface. Post treating includes the steps of porous treatment (or equivalently porous induction) at a high pH or at a low pH, followed by rinsing and drying the porous-treated film, followed by crosslinking. After crosslinking, the surface of the crosslinked film is exposed to a vapor of a fluorine compound in a simple chemical vapor deposition process (CVD). The fluorine applied by CVD gives hydrophobic or super hydrophobic properties to the surface of the thin film.

The PEM thin films made in the methods disclosed herein are industrially useful in a variety of applications, owing to their unique structures. In one embodiment, a method of filtering water during water treatment to remove impurities involves passing water through a filter, wherein the filter comprises any of the polyelectrolyte multilayer thin films described herein, or any film made by any of the methods described herein.

In other embodiments, a drug delivery system is provided that comprises a PEM thin film described herein or made by any of the methods described herein. The thin film comprises a pore and there is an active pharmaceutical agent disposed in the pore.

The structure of the PEM thin films and methods for making them are now described with reference to the figures.

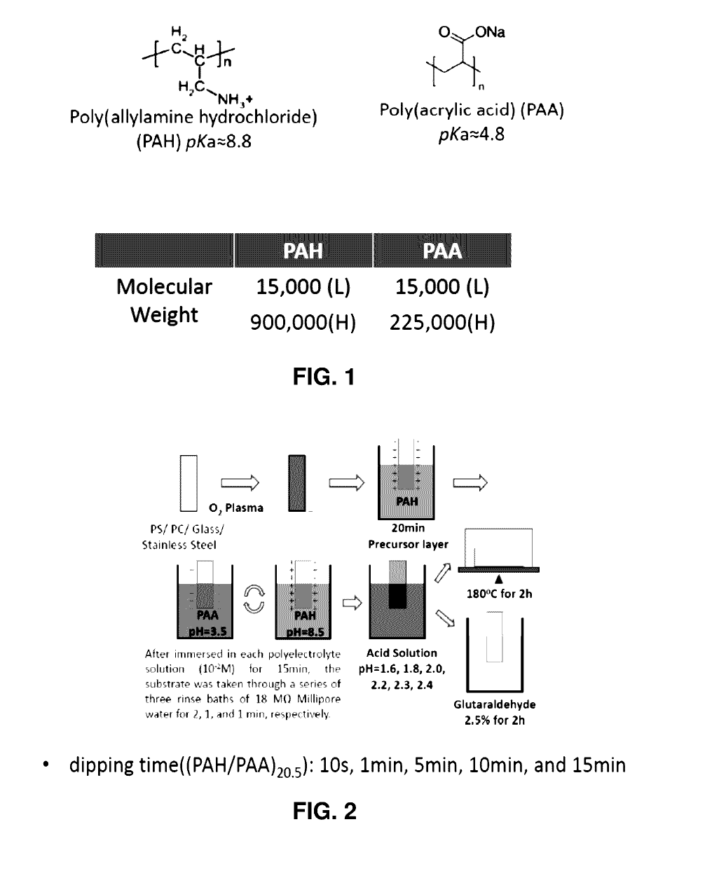

FIG. 1 provides a structure of a typical polycation, i.e. poly(allylamine hydrochloride). Polyacrylic acid is also shown, being exemplary of polyanions useful in this work. As given in the table of FIG. 1, a low molecular weight PAH has weight average molecular weight of about 15,000 g/mol, while a high molecular weight version has a molecular weight of 900,000 g/mol (unless described otherwise, all molecular weights referred to herein are weight average, and the units are g/mol). Also in FIG. 1, a low molecular weight polyacrylic has a molecular weight of 15,000, while a high molecular weight polyacrylic acid has a molecular weight of about 225,000. FIG. 2 illustrates a general process for a layer by layer (LbL) fabrication of the PEM thin film. A substrate such as polystyrene, polycarbonate, glass or stainless steel is treated, for example with oxygen plasma to give it a surface negative charge. The negatively charged surface of the substrate is then dipped in a solution of a polycation, here illustrated as PAH. Then, the substrate is alternately dipped in polyanion and polycation, as shown until the thickness of the film is built up. When the thin film has been deposited by alternately dipping in the polycation and polyanion, the thin film is then subjected to a porous treatment. by dipping into an acid solution. Porous treatment is also referred to as porosity induction.

In one embodiment, porous treatment involves exposing the thin film to a low pH solution, such as by dipping or immersion. The solution has a pH below the pKₐ of the polyanion used in making the film. For polyacrylic acid and other polymers containing carboxylic groups, the pKₐ is on the order of 4.2. in various embodiments, porous treatment is carried out at a pH that is one unit below or about two units below the pKₐ. In illustrative embodiments, porous treatment is carried out at a pH of 1.6, 1.7. 1.8, 1.9, 2.0, 2.1, 2.2, 2.3, or 2.4. In some embodiments, porous treatment is carried out within a pH range of about 1.8 to about 2.6.

In another embodiment, porous treatment involves exposing the thin film to a high pH solution. The pH is above the pK_{b} of the polycation used to make the thin film. In non-limiting fashion, the pH of the high pH solution is at least one unit higher or at least two units higher than the pK_{b}. For example, the pH is in the range of 10 to 12, 10-11, 11-12, and so on.

A porous structure is formed after the post treatment, which includes which includes porous treatment as described, rinsing in DI water, drying and cross-linking. During post treatment, the porous-treated PEM thin film is cross-linked, for example by dipping in a glutaraldehyde solution or by heating at an elevated temperature such as 180° C. for about 2 hours.

A key variable of the process is the so called dipping time, measuring the amount of time that the substrate is dipped in respectively the polyanion and polycation in order to build up multiple bilayers. As illustrated in the following Figures, experimentally the dipping time was set as 10 seconds, 1 minute, 5 minutes, 10 minutes, or 15 minutes.

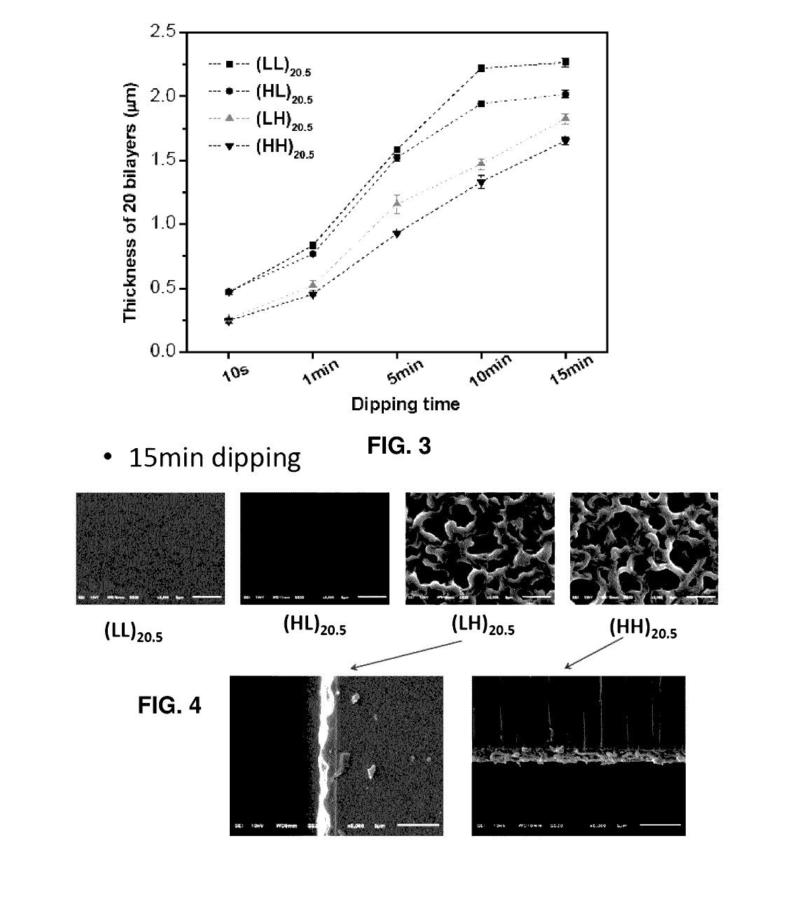

FIG. 3 shows the development of the thickness of the film according to the dipping time and as a function of the molecular weight of the respective polyanion and polycation. The general trend is that the thickness of twenty bilayers increases with dipping time. Further, the development of film thickness is different for a high molecular weight of polyanion as opposed to a low molecular weight polyanion (from FIG. 1). That is, it is observed in FIG. 3 that a thickness of 20 bilayers is greater for polyanion/polycation pairs that include a low molecular weight polyanion than it is for polyanion/polycation pairs that include a high molecular weight polyanion. To illustrate, the designation (LL)_{20.5} means 20.5 bilayers made of a low molecular weight polyanion (the letter on the right) and low molecular weight polycation (the letter on the left).

In addition to a difference in thickness illustrated in FIG. 3, the morphology of the applied PEM thin film also depends on the molecular weight of the polyanion. As shown in FIG. 4, with 15 minutes of dipping, the thin film formed using high molecular weight polyanion has a microfibrous structure. In contrast, the two samples made with low molecular weight polyanion (two micrographs on the upper left) show none of the microfibrous features. Instead, the thin films made with low molecular weight polyanion have smooth morphology. This is for an embodiment where the thin film is dipped for 15 minutes in each of the polyanion and polycation solution.

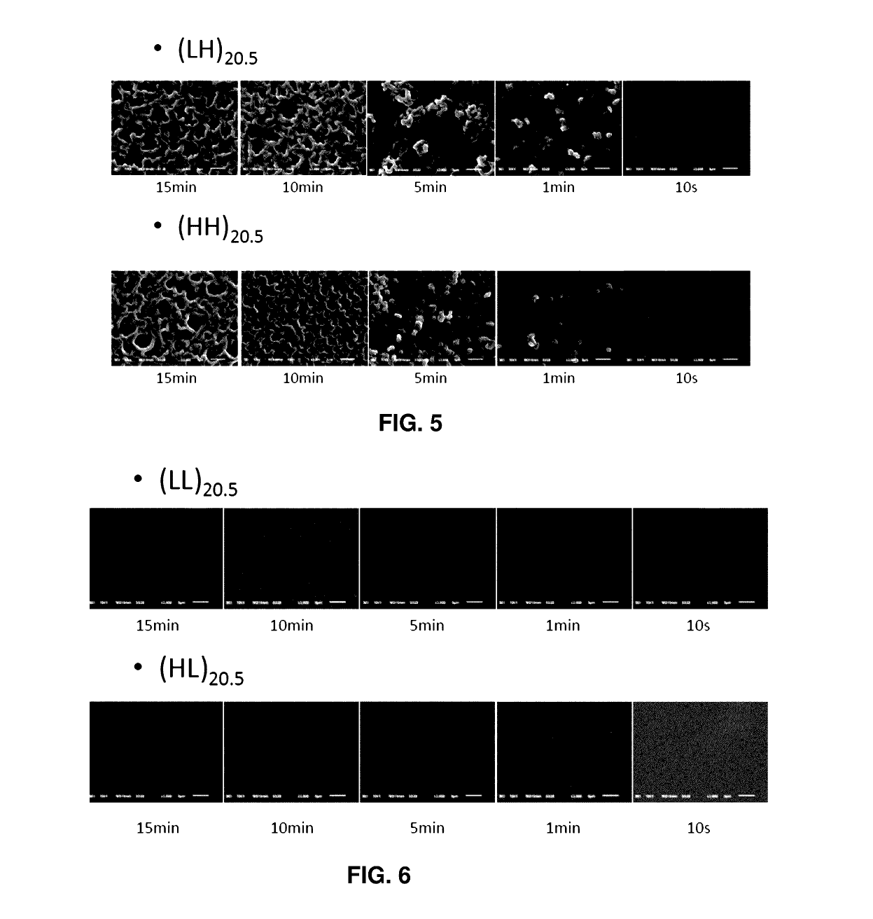

FIG. 5 illustrates the two embodiments using a high molecular weight polyanion, and shows micrographs for PEM thin films made with dipping times of 10 seconds, 1 minute, 5 minutes, 10 minutes, and 15 minutes. It is seen in FIG. 5 that at the low dipping times (e.g., 10 seconds, and 1 minute), the deposited films have a smooth morphology, whereas for longer dipping times (5 minutes, 10 minutes and 15 minutes and to a lesser extent 1 minute), the deposited thin film has a microfibrous structure.

FIG. 6 shows the contrast for the embodiments where low molecular weight polyanion is used. As shown in FIG. 6, all of the micrographs of thin films made with the low molecular weight polyanion show the same features (i.e. the smooth morphology) as the 10 second dipping times for the embodiment shown in FIG. 5 for high molecular weight polyanion.

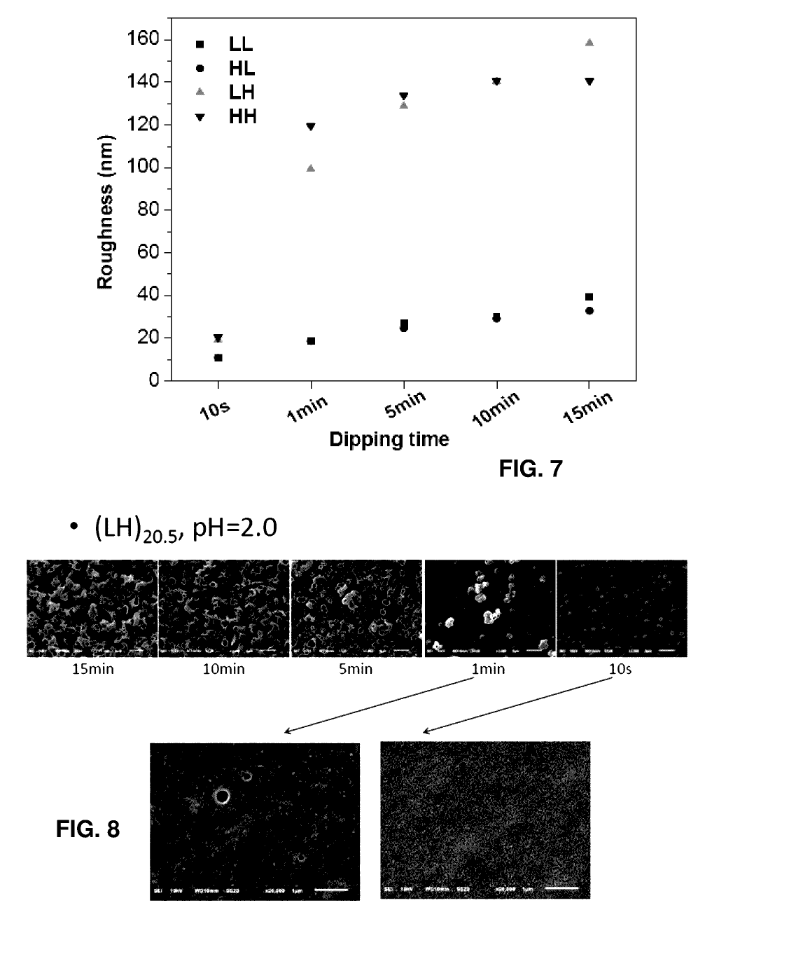

The micrographs shown in the bottom of FIG. 4 and also the graphs shown in FIG. 7 illustrate that, in addition to the difference in visual morphology of the thin films deposited using high molecular weight polyanion, the films using high molecular weight polyanion also have increased roughness.

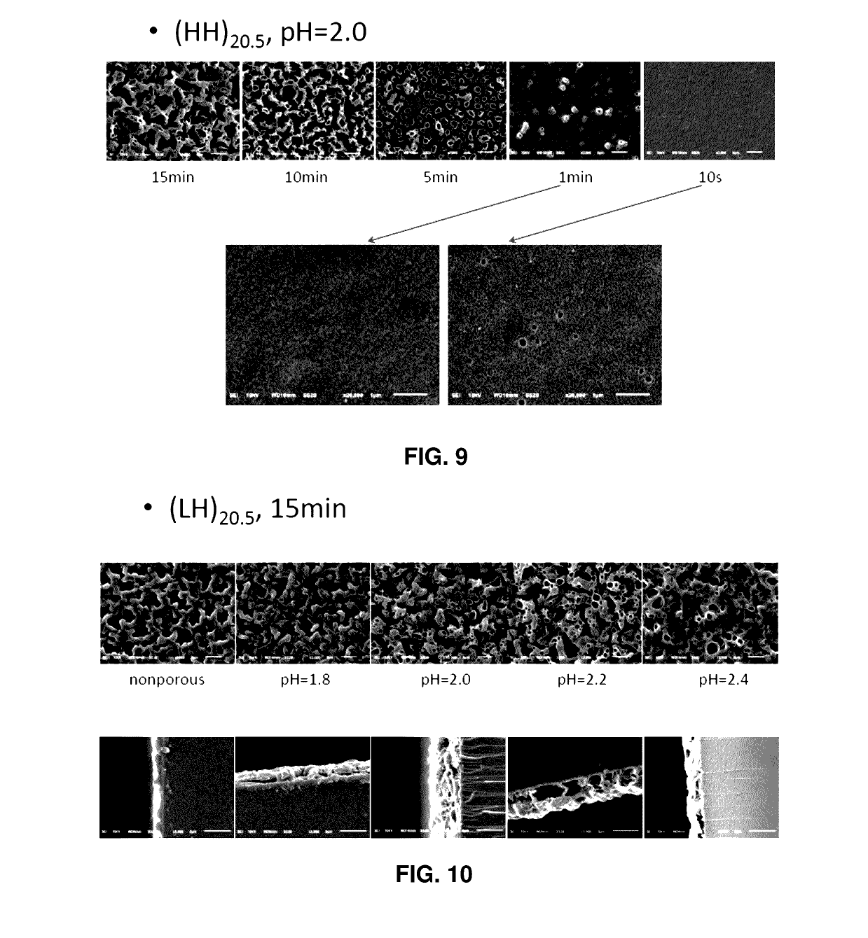

FIG. 8 shows the morphology of PEM thin films after porous treatment. The structure made by the porous treatment varies depending on the morphology produced as a function of dipping time, shown in the upper row of micrographs in FIG. 8. As shown in the Figure, the micrographs are from PEM thin films made with alternating low molecular weight polycation and high molecular weight polyanion, followed by porous treatment at pH 2.0. For the smooth morphology films produced with 10 seconds and 1 minute dipping times, the result of porous treatment shown in the bottom row is that there are pores formed on the smooth surface. At least some of the pores are nanopores in the range of 20-50 nm. On the other hand, for the microfibrous morphology produced by dipping times of 5 minutes, 10 minutes, and 15 minutes (shown in the upper row of micrographs in FIG. 8), porous treatment leads to microporous morphology. Similar results are shown in FIG. 9 for the embodiment wherein polycation was high molecular weight and polyanion was high molecular weight, with subsequent porous treatment at pH 2.0.

The result of porous treatment at different pHs is illustrated in the micrographs shown in FIG. 10. The top rows compare the morphology produced with 15 minute dipping times for the nonporous case on the left (i.e. the PEM thin film formed before porous treatment), and with porous treatment carried out at pH 1.8, 2.0, 2.2, and 2.4. The micrographs in the lower row in FIG. 10 show a side view of the thin film formed on the substrate.

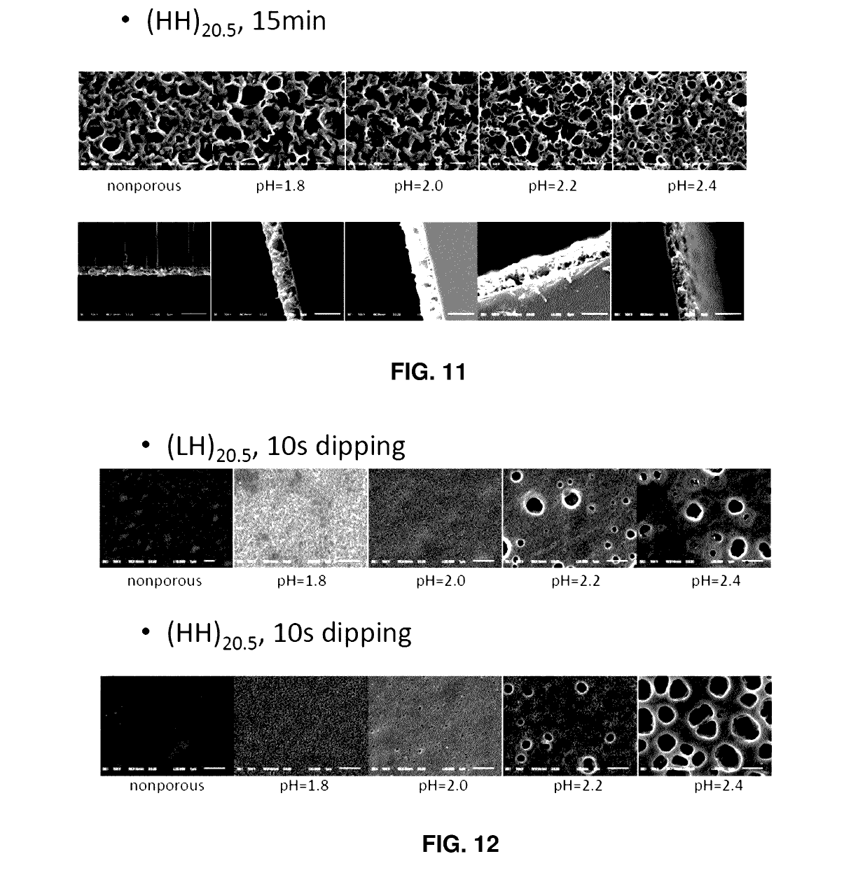

Similarly, FIG. 11 shows the results for an embodiment where the polyanion and polycation were both of the high molecular weights shown in FIG. 1. The upper row of micrographs show the microfibrous morphology generated by making the PEM thin films with 15 minute dipping time. The morphology of the nonporous thin film is compared to those thin films that are porous treated at pH of 1.8, 2.0, 2.2, or 2.4.

FIG. 12 illustrates the morphology achieved with 10 second dipping times on thin films made with high molecular weight polyanion. In the top row, the polycation was low molecular weight, while in the bottom row of micrographs in FIG. 12, the polycation was high molecular weight. The nonporous smooth morphology formed is shown in the left most micrographs of the respective rows, while the four micrographs to the right in each of the rows illustrate the morphology achieved with subsequent porous treatment at pH of 1.8, 2.0, 2.2, or 2.4. The micrographs in FIG. 12 show that porous treatment at 1.8, 2.0, or 2.2 tends to produce at least some pores in the range 20-50 nm.

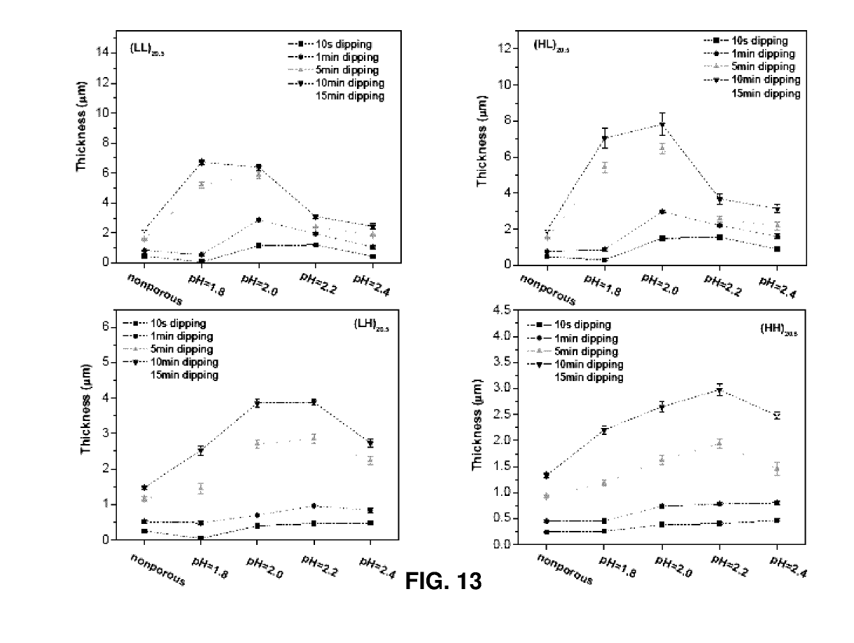

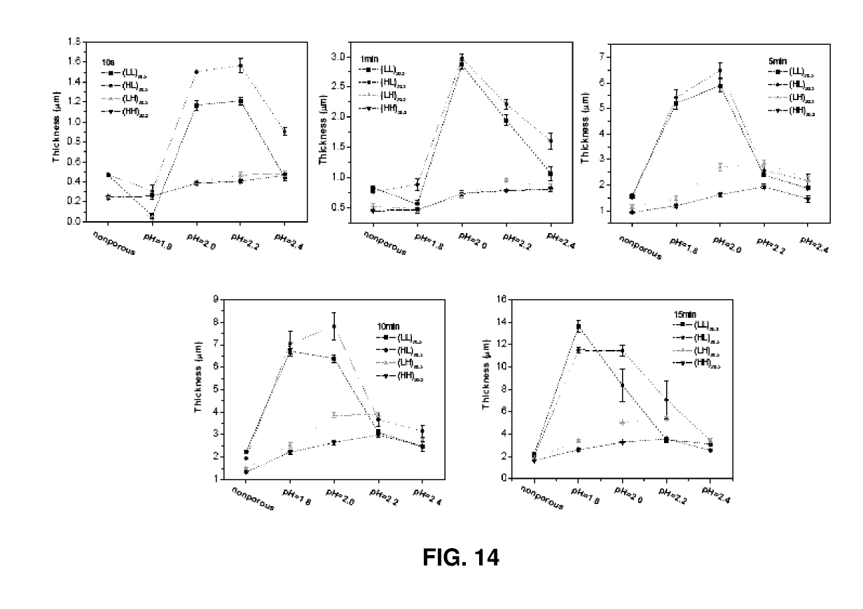

FIG. 13 shows the effect of several variables on the thickness of the PEM thin film. The variables include the dipping times (separate curves in each of the graphs), the nature of the porous treatment and the pH of the treatment (along the abscissa), and the molecular weights of the polyanion and polycation (separate graphs showing LL, HL, LH, and HH). For the bottom two graphs, wherein the polyanion was high molecular weight, it is seen that, for dipping times of 10 seconds and for 1 minute, the thickness of the film (representing 20.5 bilayers) is less than 1 micron, and is approximately the same whether the thin film is not porous treated or is treated in the range from 1.8 to 2.4. As expected, the graphs in FIG. 13 show that the thin film is thicker the longer the dipping time. The curves also show that, for dipping times of 5 minutes and greater, porous treating at the higher pHs tend to make the thin film thinner. FIG. 14 shows the same data replotted. Again it illustrates that the thickness of the PEM films formed using high molecular weight polyanion tend to be less than one micron in thickness, while other films are thicker. The graphs of FIG. 14 also show the same dependence of the thickness on the parameters of porous treatment.

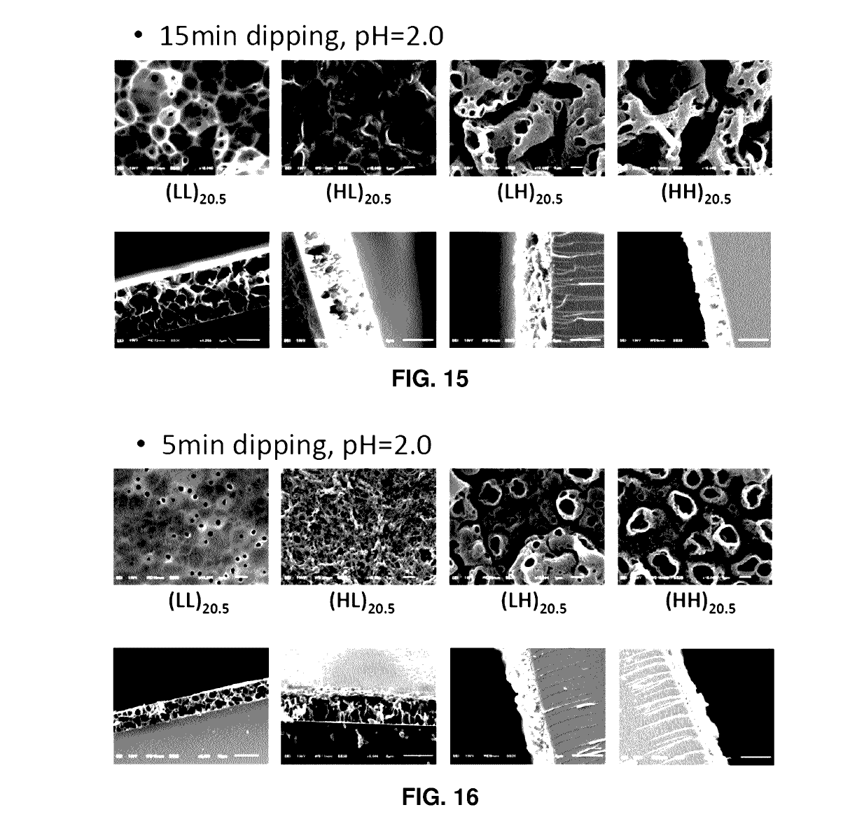

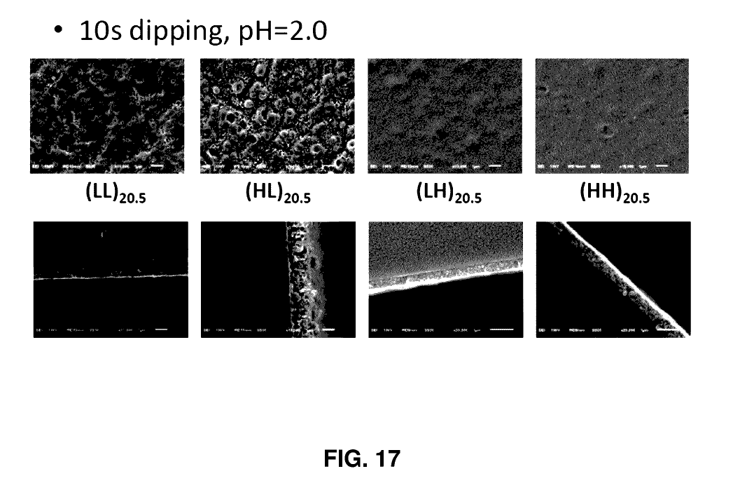

Further morphologies of films produced with different dipping times and porous treatment at various pHs are shown in FIGS. 15-17. In each of the Figures, the bottom row of micrographs shows a side view of the film developed on the substrate.

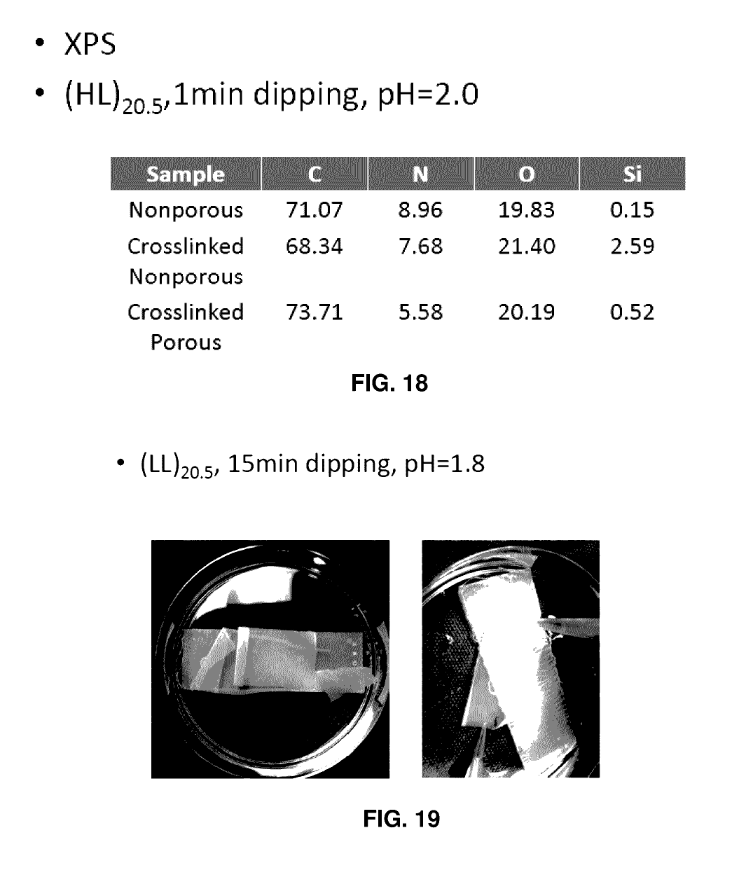

As illustrated above, after alternately applying polyanion and polycation to build up a film of the desired thickness, the thin film is optionally porous treated, after which the thin film can be further crosslinked. FIG. 18 gives the results of x-ray photoelectron spectroscopy on samples that are nonporous, that are nonporous followed by being crosslinked, and for thin film that has been porous treated followed by crosslinking. The table in FIG. 18 gives the amount of carbon, nitrogen, oxygen, and silicon in a thin film made up of 20.5 bilayers of alternating high molecular weight polycation and low molecular weight polyacrylic acid.

FIG. 19 shows what a free standing film looks like that is made with alternating layers of lower molecular weight polycation and lower molecular weight polyanion, with 15 minute dipping times, followed by a porous treatment of 1.8.

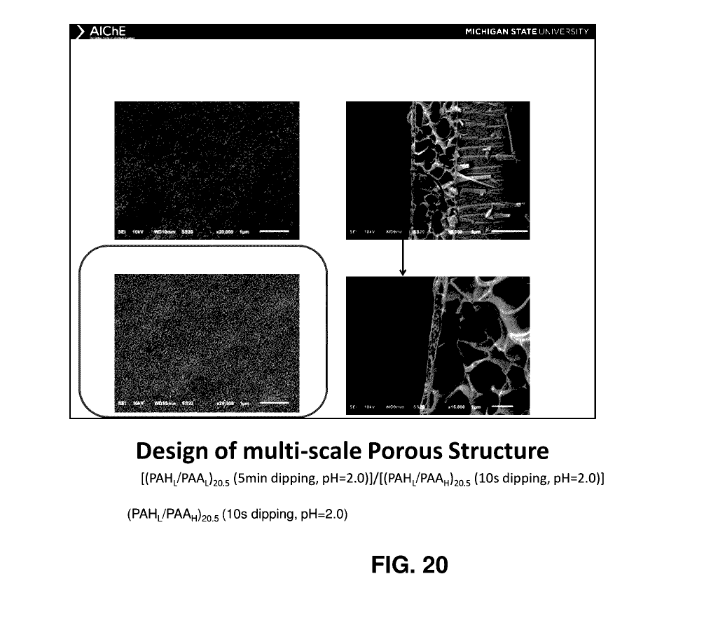

FIGS. 20-23, 25, and 27-29 illustrate various composite multiscale composites containing microporous and nanoporous morphologies.

### High Molecular Weight Polyanion

The polyanion used in the films and composites described herein is selected from one having carboxylate groups and is exemplified by polyacrylic acid. It is possible to use other polyanions, so long as their use enables the microfibrous structure observed with long dipping times and also the nanoporous structure resulting from making films with low dipping times and high molecular weight polyanion. The molecular weight in these embodiments is higher than the polyacrylic conventionally used in layer by layer construction of PEM thin films. The cutoff as to molecular weight can be determined empirically for any particular polyanion under a given set of synthetic conditions. For polyacrylic acid, it has been observed that a molecular weight above 100,000 g/mole suffices to achieve the desired morphology illustrated in the Figures. In an exemplary embodiment, the molecular weight is about 225,000 g/mole. A polyanion molecular weight at or above the value at which the microfibrous structure is formed (generally at the higher dipping times) is classified as “high molecular weight.”

### Multiscale Porous Structures

### Pores, Nanopores and Micropores

Pores in the PEM thin films described herein are classified as nanopores or micropores depending on their size. Nanopores are charactized by dimensions on the order of nanometers, and in any event less than 1 micron (which equals 1000 nm). Micropores where used indicates a pore with a dimension of 1 micron or greater. In one embodiment described herein, a film contains nanopores having a dimension of 20 nm to 50 nm. For pores that are nearly circular, such as many of the nanopores illustrated in the Figures, the word diameter can be used interchangeably for the size of the pore. But the use of “diameter” here or in the claims is not to be taken as an indication that the description is limited to round or perfectly round pores. Rather, it is a short hand way to describe the minimum dimension of a pore; if that minimum dimension is less than a micron, it is a nanopore.

The discoveries described herein have led to development of so-called multi-scale porous structures. These structures combine thin films with pores on the order of microns in size (microsized) and thin films with pores on the order of less than a micron (nanosized), for example in the range of 20-50 nm, 50-100 nm, 10-100 nm, 10-500 nm, 20-500 nm 50-500 nm, 100-500 nm, and so on. In various embodiments, a microsized porous structure is provided on top of nanosized porous structure or, conversely, a nanosized porous structure is provided on top of a microsized porous structure.

In various embodiments, a microsized porous structure as used herein is formed when a PEM thin film is built up of alternating polycation and polyanion with dipping times of greater than a minute (such as 5 minutes, 10 minutes, or 15 minutes) using the higher molecular weight polyanion. This forms the microfibrous morphology shown, for illustration, in FIG. 5. After buildup to a desired thickness, the film is subjected to porous treatment by dipping in a solution at pH 1.8-2.4, in non-limiting fashion. Conversely, a nanosized porous structure is built from depositing polycation and higher molecular weight polyanion with dipping times of about a minute or less (for example 10 seconds). When a desired thickness is reached (and remember that the thickness builds slower than with shorter dipping times), the film is again subjected to porous treatment.

Together, the microsized porous structure and the nanosized porous structure form a composite that can be applied to a variety of substrates using standard LbL technology. In a non-limiting example, a composite can be applied to a substrate comprising a non-woven fabric or non-woven web to provide membranes for various industrial applications. In non-limiting examples, they can be applied to reduce the COD level of wastewater samples. They tend to foul less than commercially available membranes. Further, the solution fluxes of the membranes are higher than commercial reverse osmosis membranes, making them less energy-demanding.

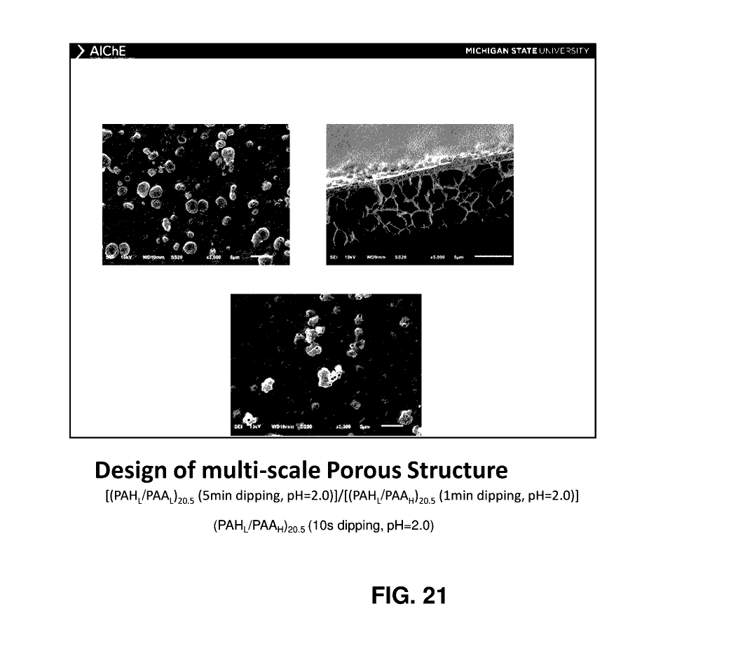

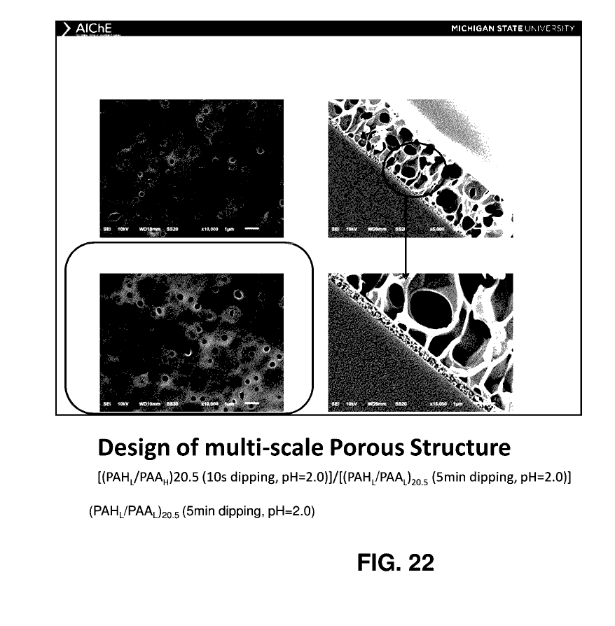

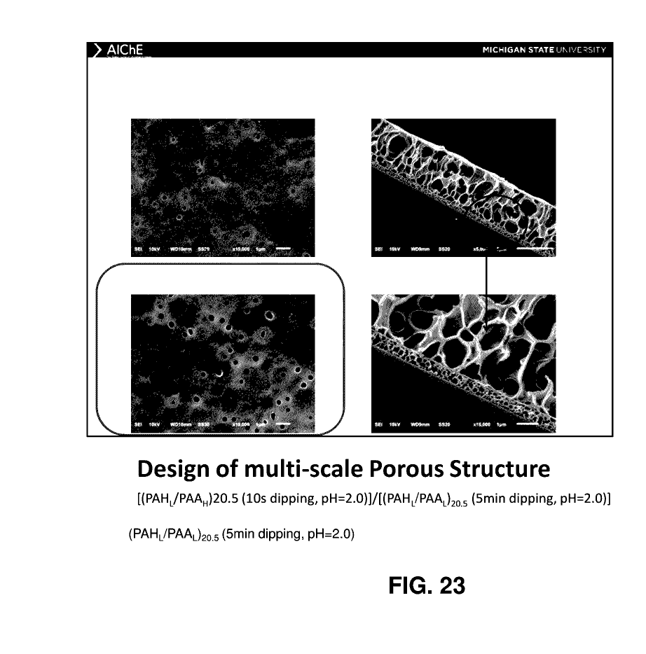

Illustrative multi-scale composites are given in FIGS. 20-23. In each Figure, micrographs are shown of the PEM film and of a cross section showing the microporous structure and the nanoporous structure. In FIGS. 20 and 21, the nanoporous structure is on top. In FIGS. 22 and 23, the nanoporous structure is on the “bottom,” i.e., it is directly disposed on the substrate. Each Figure shows the “recipe” by which the respective films were deposited. For each, the recipe for the nanoporous layer includes deposition of the high molecular weight polyacrylic (defined in FIG. 1), with a dipping time of 1 minute or less, and with porous treatment at the noted pH. On the other hand, the conditions for forming the microporous layer are likewise given: generally low molecular weight polyacrylic acid with 5 minute dipping, followed by porous treatment at the noted pH.

### Examples

### (1) Membrane Application

PEM films have been widely applied for surface modification of membranes used for water treatment applications. Commercial Ultrafiltration (UF) and Nanofiltration (NF) membranes have been modified by LbL to yield higher rejection and sometimes even higher fluxes than commercial RO membranes. However the underlying porous structures of these commercially available membranes impose certain limitations on the PEMs in terms of property enhancement. With the help of the above fabricated porous PEM structures we seek to overcome this limitation.

A commercially available NF membrane has several structural components. The bottommost support layer is usually made from non-woven PET fabrics followed by a microporous polysulfone layer. This in turn is followed by the membrane skin layer made from polyamide which usually has pores in the range of 1-5 nm. We adopt a simple bottom-up approach for mimicking the above mentioned structure using the multi-scale porous PEM structures. Both the microporous as well as the polyamide layers can be replaced by PEMs with equivalent pore sizes. The higher molecular weight of PAA can be used for fabricating the nanoporous layer with very minute pore diameters similar to what is usually observed for NF membranes. For RO applications the membrane has to be made suitable for rejecting even small monovalent ions.

This might necessitate the deposition of a few bilayers of non-porous PEMs on the nanoporous layers which would serve as a barrier to the passage of unwanted ions. Overall it can be claimed that a truly hierarchical porous structure with layers that are microporous, nanoporous and even non-porous, can be built using the simple yet versatile LbL process.

PEMs are known to be hydrophilic and by virtue of the LbL process there is control over the thickness and the pore-sizes of the fabricated membrane. This is ideal for developing a highly perm-selective membrane which should potentially eliminate the need for high pressure demands, as is presently the case. For actual desalination purposes even the best commercially available RO membranes require a transmembrane pressure of around 50-60 bar. This high pressure accounts for the lion's share of the electricity cost involved in running a desalination plant. The hierarchical porous structure provided by a multi-scale composite as described herein disposed on a porous substrate can help reduce the energy demands of the present RO membranes.

A thin-film LbL deposition technique is used to make RO membranes. In the prior art, the individual components of the membranes have to be fabricated using different processing techniques. For example, a polysulfone layer is prepared by solvent casting and a polyamide layer from interfacial polymerization. The new method described herein enables the membrane to be fabricated in a more synergistic way, whereby all the components can be synthesized using the same LbL process. This simplifies the process and saves time and money. The short-time LbL would make sure that the manufacturing of the membranes would not take long as usually expected from conventional LbL. It should also be noted that these porous structures are thermally cross-linked following the porous treatment in order to retain their structures. The cross-linking step gives the membranes mechanical strength sufficient to sustain the high pressure requirements of an RO process.

In conclusion, it can be claimed that a highly permeable RO membrane with high rejection can be made from porous LbL films. In our work we have focused on overcoming the limitation of the LbL process being time-consuming, by reducing the time of each individual step. The currently available RO membranes can be made using one simple approach without having to change the processing technique for every individual component of the membrane. Our fabrication process will not only bring down the manufacturing cost of the membranes but also the electricity cost usually required to operate these membranes in a desalination plant. Lastly, cross-linking of the multilayers would provide high mechanical strength to the membranes to be used for actual applications.

### (2) Drug Delivery

Controlled drug release from the surface has drawn more and more attention in the biomedical field. It can facilitate local delivery and increase the drug efficiency. For controlled release, the release rate should be different according to the change of drug needed for different release stage. Although LbL technique has already been applied for fabricating drug loaded PEMs (Wood K C, Boedicker J Q, Lynn D M, et al. Langmuir, 2005, 21(4): 1603-1609.), the release profile is linear for most cases, which means the release rate remains constant all the time. This is because drug release is controlled by dissociation or degradation of polyelectrolytes. In addition, this approach requires the drug to be hydrophilic, which can be alternatively deposited onto the surface with the other polyelectrolyte. For hydrophobic drug, a special amphiphilic drug carrier is required (Kim B S, Park S W, Hammond P T. Acs Nano, 2008, 2(2): 386-392.).

Dr. Rubner first developed the porous PEM films and applied for controlled drug release (Berg M C, Zhai L, Cohen R E, et al. Biomacromolecules, 2006, 7(1): 357-364.). With porous PEM films, hydrophobic drugs can be easily incorporated. The average surface pore size of their porous films ranged from 100 nm to 1 μm. In this invention, our porous structure can be precisely controlled from 20 nm to 10 μm by fast LbL assembly. What's more important, multi-scale porous structures have been successfully built up to achieve different release rate for different stage. The release kinetics can be precisely controlled by tuning the porous structure. This will help maximize the drug efficiency. The multi-scale porous structure allows us more possibility to fulfill certain needs.

### (3) Super Hydrophobic (i.e. Self-Cleaning) Coating

A super hydrophobic (i.e., self-cleaning) surface requires advancing contact angle of at least 150°. Surface roughness and topography influence the surface hydrophobicity profoundly. Micro- and nano-structure are both desired on the surface to achieve super hydrophobicity. For example, the surface of lotus leaf contains 3-10 micron-sized hills and valleys that are decorated with nano-sized hydrophobic particles. Current studies (Zhai L, Cebeci F C, Cohen R E, et al. Nano Letters, 2004, 4(7): 1349-1353.) generated super hydrophobic surface always by fabricating micro-sized structure first and then introducing nano-sized structure. With our approach, micro and nano-structured surface can be achieved at the same time by tuning the dipping time and molecular weight of polyelectrolytes. The surface can be further modified with fluorinated silane molecules to obtain a super hydrophobic surface.

Suitable fluorinated silane molecules (also called fluoroalkyl silanes) contain a perfluorinated or partially perfluorinated hydrocarbon chain attached to a silane group functionalized to bond to functional groups like hydroxyls found on the surface. A non-limiting example is trichloro-(1H, 1H, 2H, 2H-perfluorooctyl)-silane, which is commercially available from Aldrich. After post treatment, the porous surface is exposed to a fluorinated silane or other suitable molecule in the gas phase using a simple chemical vapor deposition process. For example, several droplets of the silane are placed in an open vial sitting next to the samples to be coated. The vial and samples are placed in a beaker and the beaker was sealed and placed in an oven. Heating is carried at 130° C. for 2 hours and then at 180° C. for 2 hours. The result is a hydrophilic surface covered with sufficient silane to increase the observed contact angle of water with the surface. Coverage of the fluorinated silane on the surface of the sample is inferred from the observed increase in the contact angle, and can be confirmed with an elemental analysis.

The invention has been described with exemplary embodiments based on a combination of a variety of features, each of which can take various values. It is to be understood that the various values of the features can be substituted to provide other embodiments. A non-limiting set of embodiments includes:

1. A polyelectrolyte multilayer thin film having pores in the film, wherein at least some of the pores have a diameter of 20 to 50 nm.

2. The film of embodiment 1, wherein the film comprises alternating layers of polycation and polyacrylic acid.

3. The film of embodiment 2, wherein the polyacrylic acid has a weight average molecular weight over 100,000 g/mole.

4. The film of embodiment 2, wherein the polyacrylic acid has a weight average molecular weight of about 225,000 g/mole.

5. The film of embodiment 2, wherein the polycation comprises poly(allyamine hydrochloride).

6. The film of embodiment 1, wherein the film has a thickness of 250-500 nm.

7. The film of embodiment 1, comprising high molecular weight polyanion.

8. A polyelectrolyte multilayer thin film, having a hydrophobic surface characterized by a contact angle with water of greater than 150°.

9. The film of embodiment 8, wherein the contact angle is 160° or greater.

10. The film of embodiment 8, wherein the film comprises alternating layers of polycation and of polyacrylic acid.

11. The film of embodiment 10, wherein the polycation comprises polyethleneimine, poly(allylamine hydrochloride), or DADMAC.

12. The film of embodiment 10 wherein the polyacrylic acid has a weight average molecular weight greater than 100,000.

13. The film of embodiment 10 wherein the polyacrylic acid has a weight average molecular weight of about 225,000 g/mole.

14. The film of claim 8, comprising high molecular weight polyanion.

15. A polyelectrolyte multilayer thin film comprising built up alternating layers of polycation and polyanion, wherein the polyanion comprises poly acrylic acid having a weight average molecular weight of greater than 100,000 g/mole.

16. The polyelectrolyte multilayer thin film according to embodiment 15, wherein the polyacrylic acid has a weight average molecular weight of about 225,000 g/mole.

17. The polyelectrolyte multilayer thin film according to embodiment 15, wherein the film has a smooth morphology.

18. The polyelectrolyte multilayer thin film according to embodiment 15, wherein the film has a microporous morphology.

19. The film of embodiment 15, having nanopores with a diameter in the range of 20 to 50 nm.

20. The film of embodiment 20, comprising high molecular weight polyanion.

30. A method of making a polyelectrolyte multilayer thin film using layer by layer assembly of alternating polycation and polyanion, the method comprising applying a first layer of polycation to a negatively charged substrate, and building up alternating layers to make the thin film by alternately dipping the coated substrate in a solution containing polyanion and a solution containing polycation, wherein the dipping time in each solution is one minute or less, and wherein the polyanion comprises high molecular weight polyanion or polyacrylic acid having a weight average molecular weight above 100,000 g/mole.

31. The method according to embodiment 30, comprising building up at least 10 bilayers comprising polyanion and polycation.

31. The method according to embodiment 30 or 31, further comprising porosity treating the thin film by exposing it to a solution having a pH of 1.8-2.4.

32. The method according to embodiment 31, wherein the solution has a pH of 1.8-2.2.

33. The method according to embodiment 30 through 32, wherein the dipping time is 30 seconds or less.

34. The method according to embodiment 33, wherein the dipping time is about 10 seconds.

35. The method according to embodiment 30 to 34, wherein the polyacrylic acid has a weight average molecular weight of about 225,000 g/mole.

36. The method of any of embodiments 30-35, further comprising crosslinking the thin film.

40. A method of making a polyelectrolyte multilayer thin film using layer by layer assembly of alternating polycation and polyanion, the method comprising applying a first layer of polycation to a negatively charged substrate, and building up alternating layers to make the thin film by alternately dipping the coated substrate in a solution containing polyanion and a solution containing polycation, wherein the dipping time in each solution is greater than one minute, and wherein the polyanion comprises polyacrylic acid having a weight average molecular weight above 100,000 g/mole.

41. The method according to embodiment 40, wherein the molecular weight is about 225,000 g/mole

42. The method according to claim 40, further comprising porous treating the thin film by exposing it to a solution of pH 1.8-2.4.

43. A method of filtering watering during water treatment to remove impurities, the method comprising passing water through a filter, wherein the filter comprises a polyelectrolyte thin film according to any of embodiments 1-20.

44. A method of filtering watering during water treatment to remove impurities, the method comprising passing water through a filter, wherein the filter comprises a polyelectrolyte thin film made according to the method of any of claims 30-42.

45. A method according to embodiment 43 or claim 44, wherein the method comprises reverse osmosis.

46. A drug delivery system comprising a polyelectrolyte multilayer thin film of any of claims 1-29, wherein the thin film comprises a pore and an active pharmaceutical agent disposed in the pore.

47. A composite comprising a first PEM thin film having a microsized porous structure disposed on a second PEM thin film having a nanosized porous structure.

48. The composite of embodiment 47, wherein one or both of the first and second PEM thin films comprise polyacrylic acid.

49. A membrane comprising a composite according to embodiment 47 disposed on a porous substrate.

50. The membrane of embodiment 49, wherein the porous substrate is a non-woven web.

51. The composite of embodiment 48, comprising polyacrylic acid having a weight average molecular weight of greater than 100,000 g/mole.

52. A method according any of embodiments 30 through 42, further comprising applying a fluoroalkyl silane to the surface of the thin film following post treatment, wherein the post treatment comprises porous treatment, rinsing, drying, and crosslinking.

53. The method of embodiment 52, wherein the fluoroalkyl silane comprises trichloro-(1H, 1H, 2H, 2H-perfluorooctyl)silane.

### Examples

In this work, we focused on the design of porous polymeric films with nano and micro sized pores existing in distinct zones. The porous thin films were fabricated by the post treatment of layer-by-layer (LbL) assembled poly(allylamine hydrochloride) (PAH)/poly(acrylic acid) (PAA) multilayers. In order to improve the processing efficiency, the dipping time was shortened to −10 s. It was found that fine porous structures could be created even by significantly reducing the processing time. The effect of using polyelectrolytes with widely different molecular weights was also studied. The pore size was increased by using the high molecular weight PAH, while high molecular weight of PAA minimized the pore size to nanometer scale. Having gained a precise control over the pore size, layered multi-scale porous thin films were further built up with either micro-sized porous zone on top of nano-sized porous zone or vice versa.

Porous polymeric films are in demand for a wide range of applications including foams[1], insulators[2], membranes[3], catalytic supports[4], anti-reflection coatings[5], superhydrophobic coatings[6] and drug delivery systems[7]. For many applications, sophisticated porous structures with a precise control on the pore sizes ranging from nano to micro are desired. For example, hierarchical (i.e., micro and nano sized) porous surfaces also help achieve superhydrophobicity[8]. For controlled drug release, the release rate is highly dependent on the pore size[9]. A well-controlled porous structure enables a tunable drug release over time[10]. In addition, commercial nanofiltration/reverse osmosis membranes have an asymmetric structure with two distinct types of porous zones; the bottom one consists of micro-sized pores while the upper zone consists of nano-sized pores. Motivated by these prospects, the multi-scale porous thin films with well-defined micro and nano-sized porous regions were developed in this work.

Layer-by-layer (LbL) assembly is considered as a highly versatile deposition technique for fabricating functional thin films and coatings[11]. LbL assembled polyelectrolyte multilayers (PEMs) followed by simple post-treatment steps provides one of the most promising methods to generate porous polymeric frameworks. Rubner and coworkers first demonstrated the formation of porous networks using poly (allylamine hydrochloride) (PAH)/poly (acrylic acid) (PAA) multilayers. The PEMs were fabricated with the PAH solution at a pH of 7.5 or 8.5 and the PAA solution at a pH of 3.5. The porous structure was formed after the post treatment, which includes acidic immersion within the pH range of 1.8-2.6, rinsing in DI water, drying and cross-linking[5-6, 9, 12]. Both nano and micro sized porous films were able to be achieved by tuning the post treatment conditions. In addition, free standing porous PAH/PAA films can be obtained through an ion-triggered exfoliation method[12d]. Porous thin films can also be fabricated by salt-induced structural changes in PAH/PAA multilayers[13]. The porous structures were formed by exposing the PAA/PAH multilayers fabricated in the presence of salt to pure water. However, the pore size was limited to the nanometer scale. Another way to make porous thin films via LbL assembly is through the treatment of hydrogen-bonded poly(4-vinylpyridine) (PVP)/PAA multilayers in aqueous solution at pH of 12.5 when PAA was dissolved followed by the reconfirmation of PVP chains[14]. Only micro-sized pores were obtained, and the stability of the hydrogen bonded LbL films over a broad range of pH is always an issue. Thus, in this work, we applied acidic treatment to induce the porous formation in PAH/PAA multilayer films.

Immersion of PAH/PAA films in a low-pH aqueous solution causes rearrangement of the polymer chains[5, 12b, 12c]. This rearrangement is induced by the breakage of the ionic cross-links of PAA due to protonation of the carboxylate groups and charge repulsion among the free, positively charged amine groups of PAH. The rinsing step with DI water allows ion pairs to reform and form small water pocket by rejecting water from the film. By drying water out from the water pockets and cross-linking the polymer chains, stable porous films were obtained.

In order to create distinct zones with different scales of the pore size, the pre-requisite was to have a very precise control on each of the zones independently and to understand the factors that affect the formation of those zones. Once those factors were identified, their combination could lead us to form multi-scale porous frameworks. Previous studies mainly investigated the effect of the number of layers[12a, 12d], pH[5, 9, 12a, 12c, 15] and time[12a, 12d, 15a] of the post treatment on the morphology of the porous PAH/PAA films. However, one major obstacle in commercializing any of these films is the long processing time that goes into fabricating the PAH/PAA films using LbL technique. Recently, several studies initiated using short dipping time to address this issue and apply for gas barrier films[16]. The dipping time was shortened from conventional 15-20 min to less than 1 min. It has been found that different dipping time leads to varied film compositions and structures[16a]. Since the formation of porous PAH/PAA films is mainly dependent on the interaction between PAA and PAH and the reorganization of polymer chains, the changes in film composition and polymer distribution may further alter the porous structure. However, no research has been focused on studying the effect of dipping time on the porous structure, or how efficiently the porous thin films can be built up. In addition, the mobility of the individual polymer chains also plays a crucial role. During the post treatment, the reorganization of polymer chains is highly influenced by the chain mobility and the interaction among functional groups. In this regard, the molecular weight of polyelectrolytes could be one of the critical and intrinsic parameters to tune the porous structure since it highly affects the chain mobility and the intramolecular and intermolcular interactions. It has been reported that molecular weight of polyelectrolytes plays an important role during LbL assembly[17]. However, few studies focused on the molecular weight effect of polyelectrolytes on the porous structure[15b]. In order to study the molecular weight effect thoroughly, we fabricated PAH/PAA multilayers using PAA with molecular weight of 15,000 g/mol (PAA_{L}) and 225,000 g/mol (PAA_{H}) and PAH with molecular weight of 15,000 g/mol (PAH_{L}) and 900,000 g/mol (PAH_{H}). In this study, we focused on the effect of dipping time and molecular weight of polyelectrolytes on the porous morphology in order to shorten the fabrication time and obtain a wider and more precise control on the porous structure at the same time.

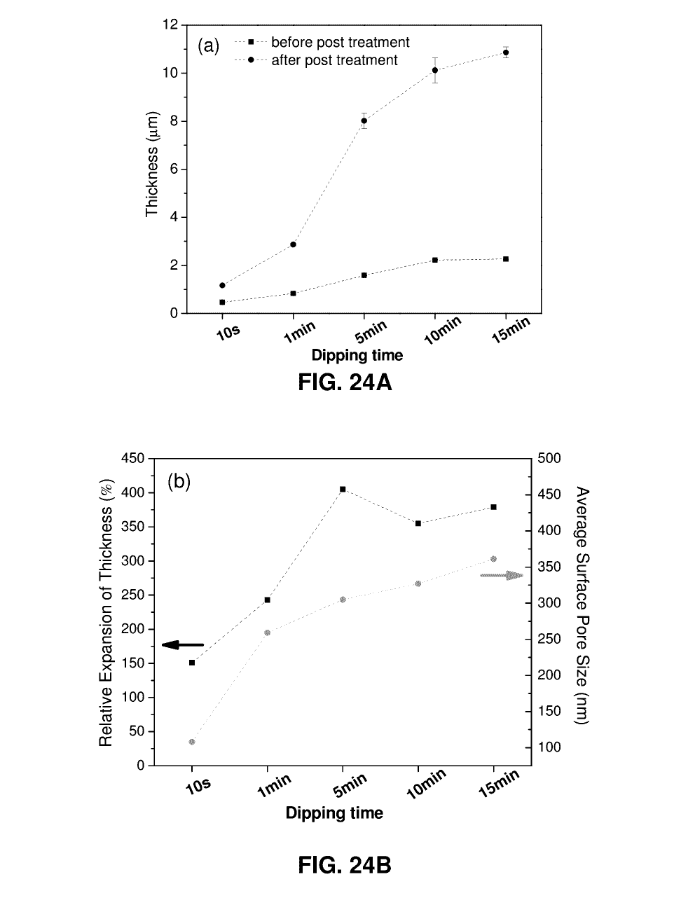

In this work, the PAH/PAA multilayers were constructed by the alternate deposition of PAH at pH=8.5 and PAA at pH=3.5. According to the literature, the degree of ionization of PAA in aqueous solution with pH=3.5 is less than 10%[18], while the degree of ionization of PAH in aqueous solution with pH=8.5 is around 50% [18a, 19]. Under this pH condition, a high level of interlayer diffusion occurs in order for charge compensation to take place, leading to an exponential growth in the thickness of the multilayer films[18b, 20]. The variations in the thickness of (PAH_{L}/PAA_{L})20.5 films as a function of the dipping time are shown in FIG. 24(a). The dipping time varies from 10 s to 15 min. Before the post treatment, the multilayer thickness increases with the increase in dipping time as a result of time-dependent interdiffusion process. For short dipping time, the interlayer diffusion is suppressed, leading to thinner multilayers. This result is consistent with several previous studies[16a, 20]. When the dipping time was increased from 10 to 15 min, the thickness almost remained the same. In fact, besides the change in thickness, the composition and distribution of PAA and PAH in the multilayers were also altered by dipping time[16a]. All these factors affect the breakage of the ionic cross-links and the rearrangement of polymer chains, leading to different porous structures. The acid treatment was carried out under the condition of pH=2.0 for 5 min followed by 5 min of washing step with DI water. FIG. 25 shows the SEM images of the surface and cross-section of the porous structure with different dipping time. The values of average surface pore size are summarized in FIG. 24(b). The average surface pore size increased sharply from approximately 108 to 259 nm when the dipping time changed from 10 s to 1 min. With dipping time further increased to 5, 10 and 15 min, the average surface pore size increased to approximately 305, 327 and 361 nm, respectively. In general, longer dipping time created larger surface pore size. It is also obvious from FIG. 25 that the inner pore size is different than the surface pore size. Micro-sized pores were successfully formed throughout the entire cross-section of the films. It is hard to measure the actual inner pore size, because the pores were highly interconnected. However, it is still obvious that the inner pore size increased as the dipping prolonged from 10 s to 1 min. FIG. 24(b) also shows the relative expansion of thickness as a function of dipping time. The value is not always proportional to the dipping time. This indicates that several intertwined factors like film composition, polyelectrolyte distribution, the nature of polyelectrolytes (i.e., chain mobility, hydrophilicity), mass lost during the post treatment[15a], etc., influence the porous structure in a synergistic manner. The interlayer diffusion during LbL assembly definitely facilitated the formation of pores. Even though there are certain differences in thickness and pore size, the porous structures are very similar to each other when dipping time is longer than 5 min. Proper porous structure could be generated by the PAH_{L}/PAA_{L} multilayers assembled with dipping time of 10 s in a much faster way and with a smaller pore size.

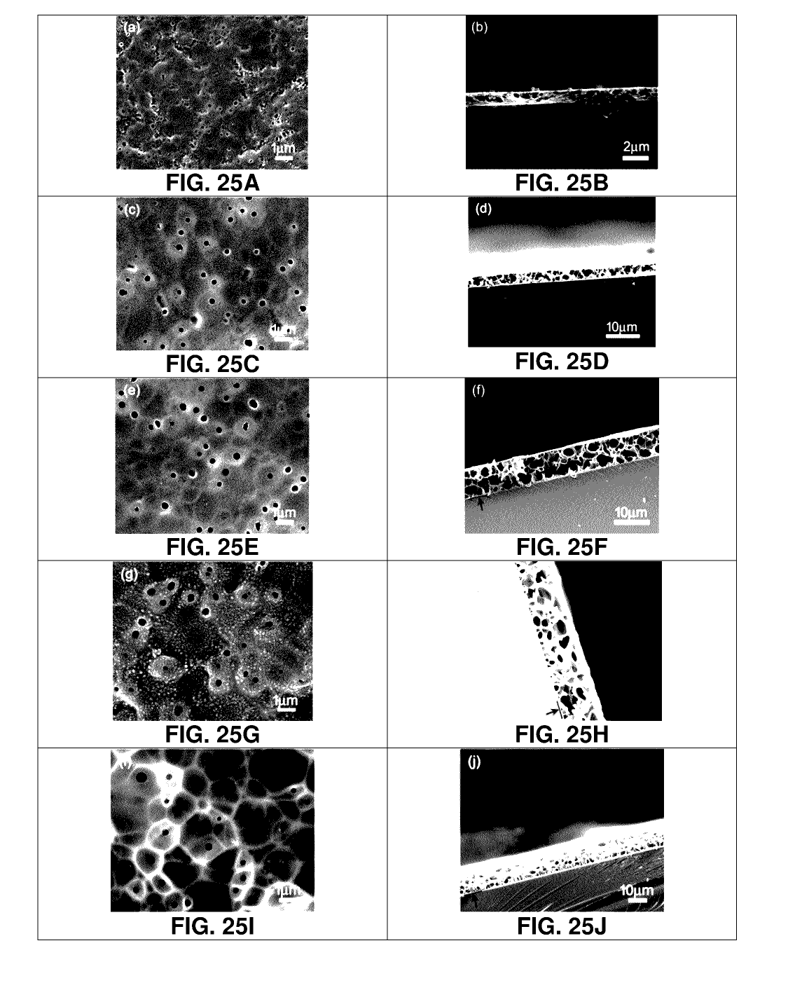

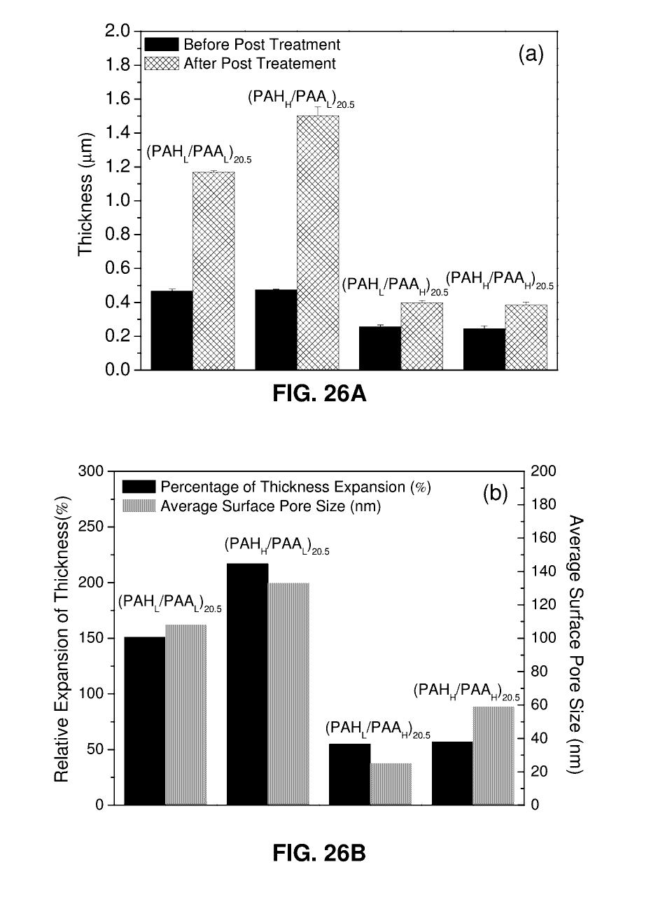

Considering the efficiency of fabricating porous films, 10 s dipping was further applied to different molecular weight systems in order to study the molecular weight effect on the porous morphology. The acid treatment was still carried out by immersing PAH/PAA multilayers in pH=2.0 aqueous solution for 5 min followed by 5 min of washing with DI water. FIG. 26(a) illustrates the effect of molecular weight on the thickness of the films before and after post treatment. Before porous treatment, the thickness for (PAH_{L}/PAA_{L})20.5 is almost the same as (PAH_{H}/PAA_{L})20.5. Similar results were also found for (PAH_{L}/PAA_{H})20.5 and (PAH_{H}/PAA_{H})20.5 films, which means the molecular weight of PAH does not affect the thickness of the films significantly. However, high molecular weight of PAA led to a decrease in thickness for multilayers fabricated using the same molecular weight of PAH. After the post treatment, the relative expansion of thickness for (PAH_{L}/PAA_{L})20.5 (PAH_{H}/PAA_{L})20.5, (PAH_{L}/PAA_{H})20.5, and (PAH_{H}/PAA_{H})20.5 are shown in FIG. 26(b), respectively. With the same molecular weight of PAA, high molecular weight of PAH provided higher relative expansion of thickness; while with same molecular weight of PAH, high molecular weight of PAA limited the thickness expansion during the post treatment. SEM images for all four porous thin films are shown in FIG. 27. It is obvious from the top-view images in FIG. 27 that high molecular weight of PAH not only creates larger surface pore size but also leads to a less uniform pore size distribution. In addition, high molecular weight PAA lowers the pore size significantly, which is consistent with what has been reported previously[15b]. The values of average surface pore size are presented in FIG. 26(b). The surface pore size was able to be tuned from 25 to 133 nm with different molecular weight combinations. Similar results can also be obtained for the inner pore size from the cross-sectional images in FIG. 27, that high molecular weight of PAA led to a decrease in the inner pore size, while high molecular weight of PAH provided larger inner pores. According to the previous studies[12b, 12c], when PAH/PAA multilayers are immersed in pH=2.0 aqueous solution, the carboxylate groups from PAA are protonated leading to the breakage of ionic cross-links, while the amine groups from PAH become fully charged. The intramolecular charge-charge repulsion for high molecular weight of PAH is much stronger than that of low molecular weight of PAH. This explains why the high molecular weight PAH caused larger pore size as well as higher relative expansion of thickness. Besides, the reorganization of polymer chain during post treatment is highly dependent on the chain mobility of the polyelectrolytes. PAA has very low charge density when immersed in pH=2.0 aqueous solution. The chain mobility is highly limited by using higher molecular weight of PAA, leading to a smaller pore size and consequently a lower relative expansion of thickness. It is apparent that the molecular weight of the polyelectrolytes plays a very important role in the formation of porous structures. However, the molecular weight effect doesn't only exist during the post treatment. During the LbL assembly, the molecular weight of polyelectrolytes also affects the adsorption and interlayer diffusion, leading to different film composition and distribution of polyelectrolytes[17b, 17d, 21]. It is hard to differentiate how these factors affect the porous structure independently, even though the dipping time of 10 s was applied when the interlayer diffusion is highly limited. But from the above results, there is no doubt that changing molecular weight of polyelectrolytes enables a wider control of pore size and morphology.

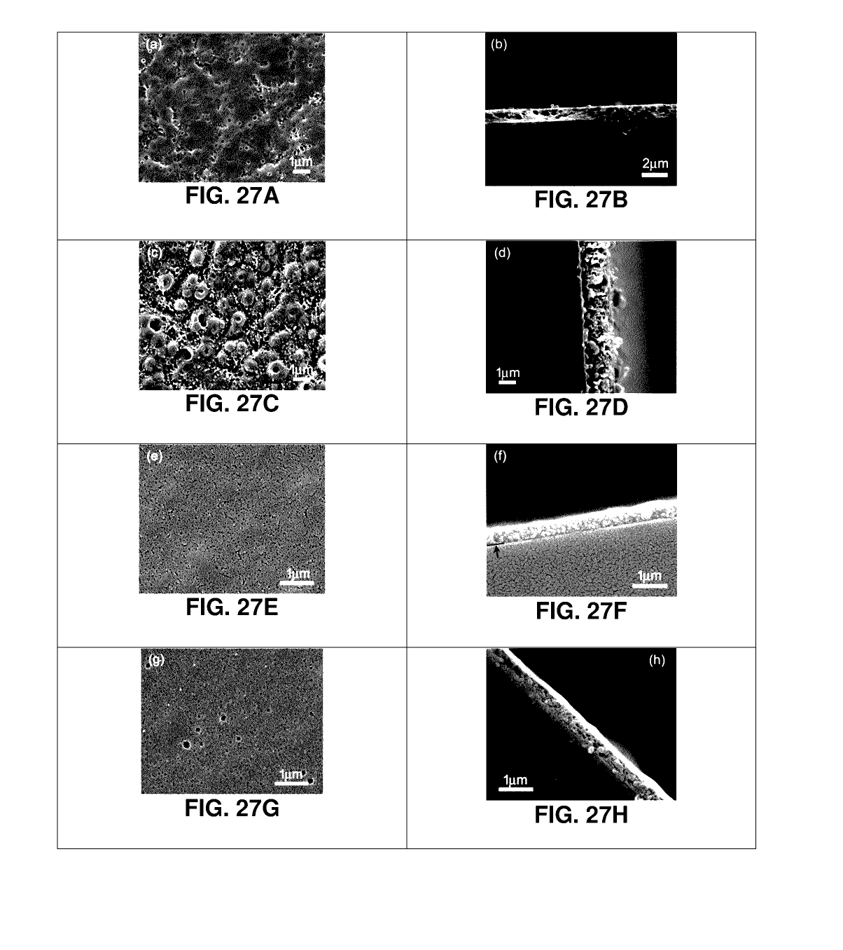

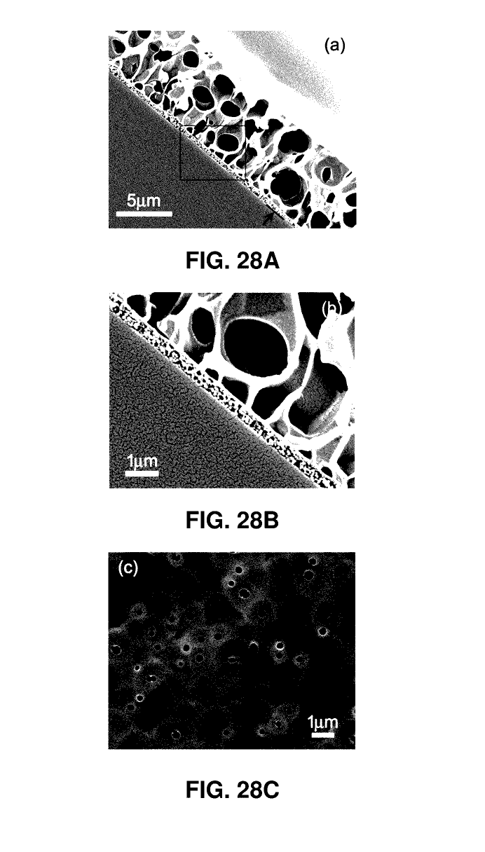

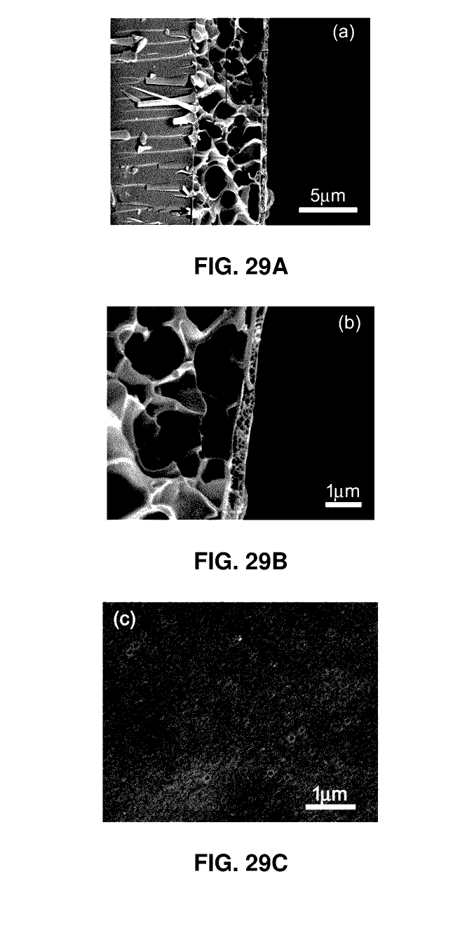

Based on the porous structures described above, multi-scale porous films were fabricated which constituted of a macro-sized porous zone on top of nano-sized porous zone (FIG. 28) or the other way around (FIG. 29). To fabricate these films, the bottom porous portion was made first using the usual protocol of LbL assembly followed by the post treatment. The PAA and PAH chains were completely reorganized during the acid immersion and DI water rinsing step, leaving both COO⁻ groups and NH₃⁺ groups on the surface. The cross-linking step causes the formation of amide bonds (—NHCO—) between the COO⁻ groups of PAA and NH₃⁺ of PAH and preserves the porous structure from being altered by further immersion in aqueous solution^{[12c,22]}. Some free carboxylate groups and ammonium groups remained in the films after the cross-linking^{[22]}. The remaining free ammonium groups on the surface enabled the deposition of PAA at pH 3.5, when ammonium groups are completely charged and carboxylate groups become mostly protonated. The bottom porous thin film thereby acted as the substrate to further build up porous films on the top. Considering the quality of the porous structure and the fabrication efficiency, porous PAH_{L}/PAA_{L} thin film with 5 min dipping was selected as the micro-sized porous portion, while porous PAH_{L}/PAA_{H} thin film with dipping time of 10 s was chosen as the nano-sized porous portion.

As shown in FIG. 28, porous (PAH_{L}/PAA_{H})_{20.5} thin film with dipping time of 10 s was first built up as the bottom portion, followed by the porous (PAA_{L}/PAH_{L})₂₀ thin film with dipping time of 5 min. Two clearly defined zones with different pore sizes were fabricated by this method, without any significant penetration of polyelectrolytes into the nano-sized bottom portion. In addition, the surface and cross sectional morphology for micro-sized top zone of the multi-scale porous films remained almost the same as the simple porous (PAH_{L}/PAA_{L})_{20.5} films with dipping time of 5 min (FIGS. 25 (e) and (f)). Hence the substrate effect was minimal for the micro-sized porous top zone.

In the above mentioned scenario, the underlying porous portion had very small surface pore size, therefore the molecular weight of the polyelectrolytes used to build up the top porous portion, is not a matter of serious concern. However, if the bottom portion is made with relatively larger surface pore size, PAA with high molecular weight is required for the top porous portion. This is because the polymer chain size needs to be large enough for not diffusing into the porous bottom. In addition, after the bottom portion was thermally cross-linked, the surface became more hydrophobic, which helped trap air inside the porous structure and block the polyelectrolytes outside. As shown in FIGS. 29 (a) and (b), nano-sized porous structure of (PAA_{H}/PAH_{L})₂₀ with 10 s dipping has been successfully fabricated on top of the porous (PAH_{L}/PAA_{L})_{20.5} films with dipping time of 5 min. It is clear that no polyelectrolytes entered into the micro-sized porous bottom. FIG. 29(c) shows the top view of this particular multi-scale porous thin film. Compared to FIG. 27(e), it has been found that the number of pores decreases, while the pore size increases to 50±19 nm. There was a slight change in the porous morphology for the nano-sized porous region of the multi-scale porous thin film from the simple porous (PAA_{H}/PAH_{L})₂₀ film with 10 s dipping. This is mainly because the micro-sized porous bottom has different charge density than the plasma treated glass slides, and the substrate effect is relatively more obvious when the film is very thin.

In summary, multi-scale porous thin films have been developed for the first time with either micro-sized porous structure on top of nano-sized porous structure or vice versa. In order to build up the porous thin films more efficiently, the effect of dipping time on the morphology of porous films was investigated for the first time in this work. Compared to conventional 15 or 20 min dipping, we were able to shorten the dipping time to 10 s but still maintain fine porous structures. The molecular weight effect of both PAH and PAA were also studied. While an increase in the molecular weight of PAH led to an increase in the pore size, a decrease in the pore size was observed for a high molecular weight of PAA. The layered multi-scale porous thin films were further fabricated by tuning the tipping time and molecular weight of polyelectrolytes. The porous thin films developed in the present work may broaden the applications of porous thin films for membrane filtration, drug delivery, etc.

### Materials

Poly (acrylic acid, sodium salt) solutions with different molecular weight (PAA_{L}, Mw=15,000, 35% aqueous solution, and PAA_{H}, Mw=225,000, 20% aqueous solution) were purchase from Sigma Aldrich and Polyscience, respectively. Both poly(allylamine hydrochloride) (PAH_{L}, Mw=15,000 g/mol and PAH_{H}, Mw=900,000 g/mol) were purchased from Sigma-Aldrich. All aqueous solutions were prepared using 18.2 MΩ Millipore water at a concentration of 10 mM with respect to the repeat unit and adjusted to the required pH using 0.1M HCl or NaOH solutions. Glass slides from Globe Scientific Inc. were cleaned by sonication for 20 min each in ethanol and DI water and then exposed to oxygen plasma generated by a Harrick plasma cleaner (Harrick Scientific Corporation, Broading Ossining, NY) for 20 min, producing hydrophilic moieties and negative charges on the surface.

### Fabrication of LbL Assembled Films and Porous Films

All LbL films were assembled with a programmable Carl-Zeiss slide-stainer. After the oxygen plasma treatment, the glass substrates were immediately dipped into PAH solution (without adjusting the pH) for 20 min to form the precursor layer, followed by three washing steps. Then, the substrates were introduced in the aqueous solution of PAA (pH=3.5) for required dipping time, followed by three washing steps with DI water (pH=3.5) for sufficient time. Subsequently, the substrates were immersed in the PAH (pH=8.5) aqueous solution with the same dipping time as PAA, and washed again three times with DI water (pH=8.5). The dipping process was repeated 20 times. In total, 20.5 bilayers were deposited on the substrate, including the first PAH precursor layer. Dipping time of 10 s, 1 min, 5 min, 10 min and 15 min were applied in this work.

The assembled polyelectrolyte multilayer films were immersed in the water solution with pH of 2.0 for 5 min followed by washing with DI water (pH=5.5) for 5 min. After the porosity induction, the films were dried and then heated at 180° C. for 2 hours to cross-link the films and prevent the porous structure from being distorted. This post treatment helped create porous films as described by other researchers.[5, 9, 12c, 15a]

For fabricating the layered multi-scale porous thin films, the bottom porous portion was first built up by repeating the previous steps and considered as the substrate for the following LbL assembly. The bottom porous portion was further introduced in the aqueous solution of PAA (pH=3.5) for required dipping time and then PAH (pH=8.5) aqueous solution with three washing steps in between. After 20 bilayers of PAA/PAH were built up, the entire thin film went through the post treatment again for making the top portion porous. Eventually, the entire thin film was further thermally cross-linked at 180° C. for 2 hours.

### Film Characterization

The thickness of the thin films before and after the post-treatment was measured in the dry state using a Dektak surface profiler. A JEOL 6610LV Scanning Electron Microscopy (SEM) was used to observe the surface and fractured cross-section morphology of the porous thin films. All specimens were coated with gold before examination under the SEM.

### REFERENCES FOR THE EXAMPLES

[1] S. K. Goel, E. J. Beckman, Polymer Engineering & Science 1994, 34, 1137-1147.
[2] C. V. Nguyen, K. R. Carter, C. J. Hawker, J. L. Hedrick, R. L. Jaffe, R. D. Miller, J. F. Remenar, H.-W. Rhee, P. M. Rice, M. F. Toney, Chemistry of Materials 1999, 11, 3080-3085.
[3] aO. Sanyal, A. N. Sommerfeld, I. Lee, Separation and Purification Technology 2015; bO. Sanyal, I. Lee, J Nanosci Nanotechno 2014, 14, 2178-2189.
[4] F. Svec, J. M. Fréchet, science 1996, 273, 205-211.
[5] J. A. Hiller, J. D. Mendelsohn, M. F. Rubner, Nature materials 2002, 1, 59-63.
[6] L. Zhai, F. C. Cebeci, R. E. Cohen, M. F. Rubner, Nano letters 2004, 4, 1349-1353.
[7] D. A. Edwards, J. Hanes, G. Caponetti, J. Hrkach, A. Ben-Jebria, M. L. Eskew, J. Mintzes, D. Deaver, N. Lotan, R. Langer, science 1997, 276, 1868-1872.
[8] J. Fu, J. Ji, L. Shen, A. Küller, A. Rosenhahn, J. Shen, M. Grunze, Langmuir 2008, 25, 672-675.
[9] M. C. Berg, L. Zhai, R. E. Cohen, M. F. Rubner, Biomacromolecules 2006, 7, 357-364.
[10] S. B. Yoon, K. Sohn, J. Y. Kim, C.-H. Shin, J.-S. Yu, T. Hyeon, Advanced Materials 2002, 14, 19.
[11] aG. Decher, science 1997, 277, 1232-1237; bR. Iler, Journal of colloid and interface science 1966, 21, 569-594; cI. Lee, Langmuir 2013, 29, 2476-2489.
[12] aC. Sung, Y. Ye, J. L. Lutkenhaus, ACS Macro Letters 2015, 4, 353-356; bK.-K. Chia, M. F. Rubner, R. E. Cohen, Langmuir 2009, 25, 14044-14052; cJ. Mendelsohn, C. J. Barrett, V. Chan, A. Pal, A. Mayes, M. Rubner, Langmuir 2000, 16, 5017-5023; dX. Chen, J. Sun, Chemistry—An Asian Journal 2014, 9, 2063-2067.
[13] A. Fery, B. Schöler, T. Cassagneau, F. Caruso, Langmuir 2001, 17, 3779-3783.
[14] aS. Bai, Z. Wang, X. Zhang, B. Wang, Langmuir 2004, 20, 11828-11832; bY. Fu, S. Bai, S. Cui, D. Qiu, Z. Wang, X. Zhang, Macromolecules 2002, 35, 9451-9458.
[15] aC. Cho, N. S. Zacharia, Langmuir 2011, 28, 841-848; bB. Sun, R. M. Flessner, E. M. Saurer, C. M. Jewell, N. J. Fredin, D. M. Lynn, Journal of colloid and interface science 2011, 355, 431-441.
[16] aD. A. Hagen, B. Foster, B. Stevens, J. C. Grunlan, ACS Macro Letters 2014, 3, 663-666; bY.-H. Yang, F. A. Malek, J. C. Grunlan, Industrial & Engineering Chemistry Research 2010, 49, 8501-8509.
[17] aS. T. Dubas, J. B. Schlenoff, Macromolecules 2001, 34, 3736-3740; bC. Porcel, P. Lavalle, G. Decher, B. Senger, J.-C. Voegel, P. Schaaf, Langmuir 2007, 23, 1898-1904; cY. Jang, J. Seo, B. Akgun, S. Satija, K. Char, Macromolecules 2013, 46, 4580-4588; dB. Sun, C. M. Jewell, N. J. Fredin, D. M. Lynn, Langmuir 2007, 23, 8452-8459.
[18] aJ. Choi, M. F. Rubner, Macromolecules 2005, 38, 116-124; bN. S. Zacharia, M. Modestino, P. T. Hammond, Macromolecules 2007, 40, 9523-9528.
[19] H. Ochiai, Y. Anabuki, O. Kojima, K. Tominaga, I. Murakami, Journal of Polymer Science Part B: Polymer Physics 1990, 28, 233-240.
[20] P. Bieker, M. Schonhoff, Macromolecules 2010, 43, 5052-5059.
[21] P. Kujawa, P. Moraille, J. Sanchez, A. Badia, F. M. Winnik, Journal of the American Chemical Society 2005, 127, 9224-9234.
[22] J. J. Harris, P. M. DeRose, M. L. Bruening, Journal of the American Chemical Society 1999, 121, 1978-1979.

### Super Hydrophobic Surfaces

Porous induction was carried out after the LbL assembly of PAA (pH 3.5) and PAH (pH 8.5). The assembled polyelectrolyte multilayer films were porous treated by immersing in a water solution at pH=2.0 for 5 min followed by washing with deionized water (pH=5.5) for 5 min. After the porous induction, the films were dried and then heated at 180° C. for 2 hours to cross-link the films and prevent the porous structures from being distorted. In the end, a chemical vapor deposition process using trichloro-(1H, 1H, 2H, 2H-perfluorooctyl)silane was carried out at 130° C. for 2 hours, followed by heating at 180° C. for 2 hours to remove free fluoroalkyl silane molecules. The effect of molecular weight and dipping time on the roughness and wettability of the porous surface was investigated.

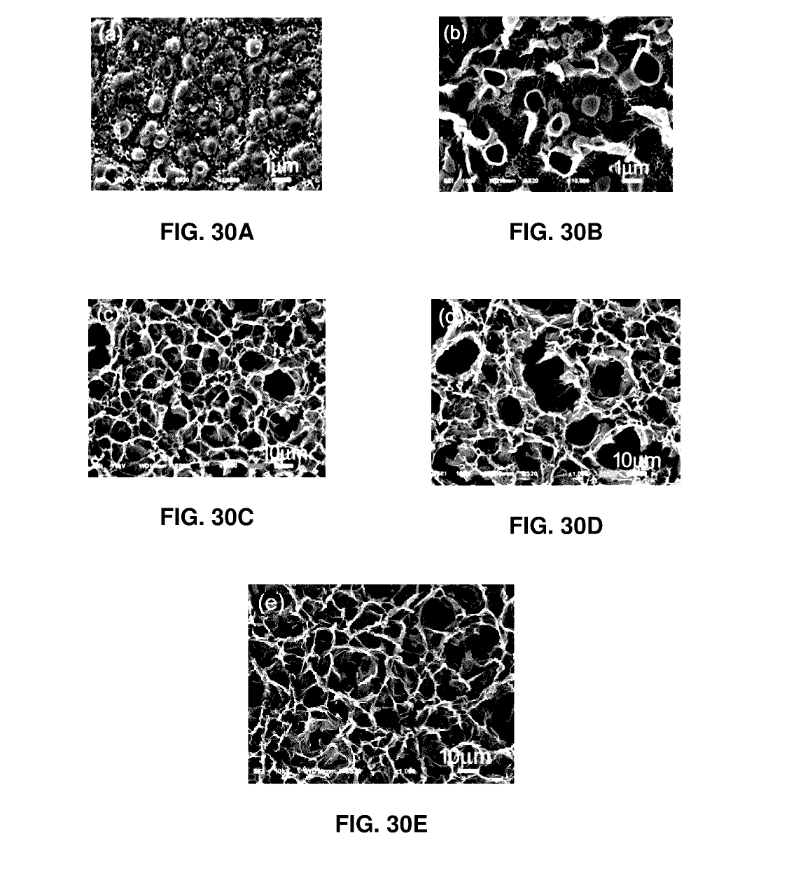

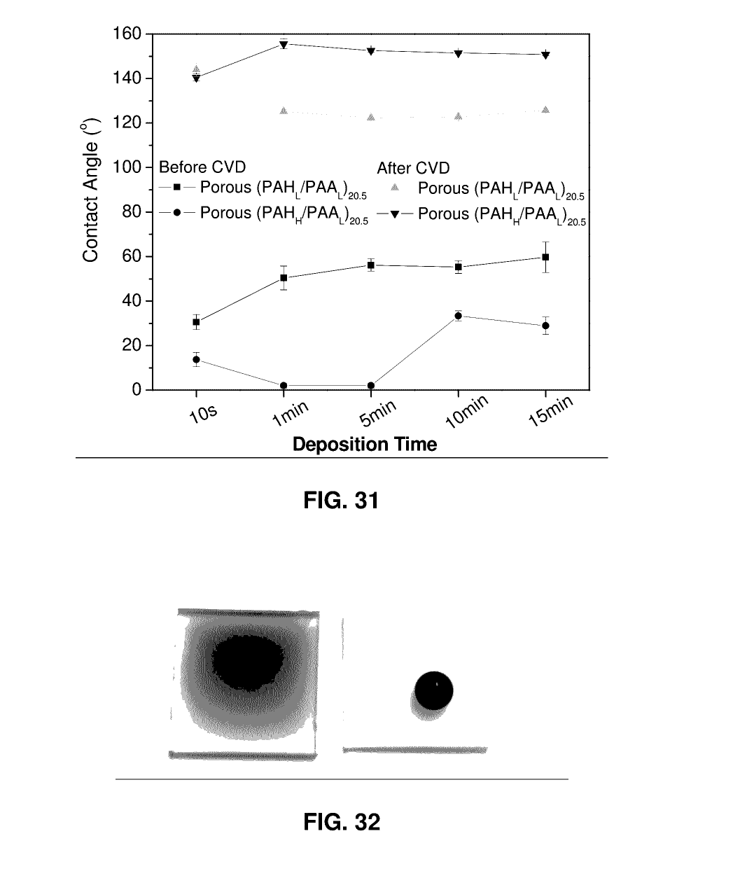

FIG. 30 includes the SEM images of porous surfaces for (PAH_{H}/PAA_{L})_{20.5}. In general, the surface roughness was increased after the porous induction. For the PAA_{L}/PAH_{L} system, the porous surface was flatter than that of (PAH_{H}/PAA_{L})_{20.5}, leading to higher contact angle before CVD process and smaller contact angle after CVD in FIG. 31. For the PAA_{L}/PAH_{H} system, it was found that the contact angle reached over 150° when dipping time increased to 1 min. With further increase of the dipping time, the contact angle slightly decreased but was still over 150°. From FIG. 30(b), it is seen that the porous induction not only enhanced the surface roughness but also facilitated the formation of hierarchical surface topography, which leads to a successful transformation from superhydrophilicity (contact angle ˜0°) to superhydrophobicity (contact angle=155.6±2.2°) through chemical vapor deposition (CVD) of fluoroalkylsilane molecules as presented in FIG. 31. Moreover, we were able to shorten the dipping time to 1 min.

A droplet of water-soluble ink solution was placed on the surfaces of the (PAH_{H}/PAA_{L})_{20.5} samples with 1 min dipping and pH=2.0 for porous induction before and after CVD process, respectively. The result is shown in FIG. 32. Before the CVD process, the surface was superhydrophilic, leading to a complete wetting by the ink droplet. After the CVD process, the droplet beaded up on the surface, which means the surface turned super hydrophobic.

In summary, by tuning the dipping time, molecular weight of polyelectrolytes and pH of the porous induction, we successfully fabricated a surface with hierarchical structure by depositing porous (PAH_{H}/PAA_{L})_{20.5}. A switch from super hydrophilicity to super hydrophobicity was achieved via a simple chemical vapor deposition of fluoroalkylsilane molecules. And it was possible to shorten the dipping time from conventional 15 or 20 min to only 1 min.

### Example

### Super Hydrophobic Thin Films

Wettability is a fundamental property of a solid surface and plays a key role in addressing the problems related to fouling[1], oil/water separation[2], corrosion[3], fogging[4], water collection[5], etc. In order to achieve superwettability, surface chemistry and topography are the two key factors. Comparing to other surface modification methods, Layer-by-Layer (LbL) assembly can be carried out under much milder conditions and provides highly tunable surface properties. LbL assembly always provides hydrophilic surface due to the nature of polyelectrolytes. Fluoroalkylsilane molecules can be grafted onto the surface by chemical vapor deposition (CVD) and change the surface wettability to hydrophobic. However, without proper surface topography, it is hard to achieve superwettability.

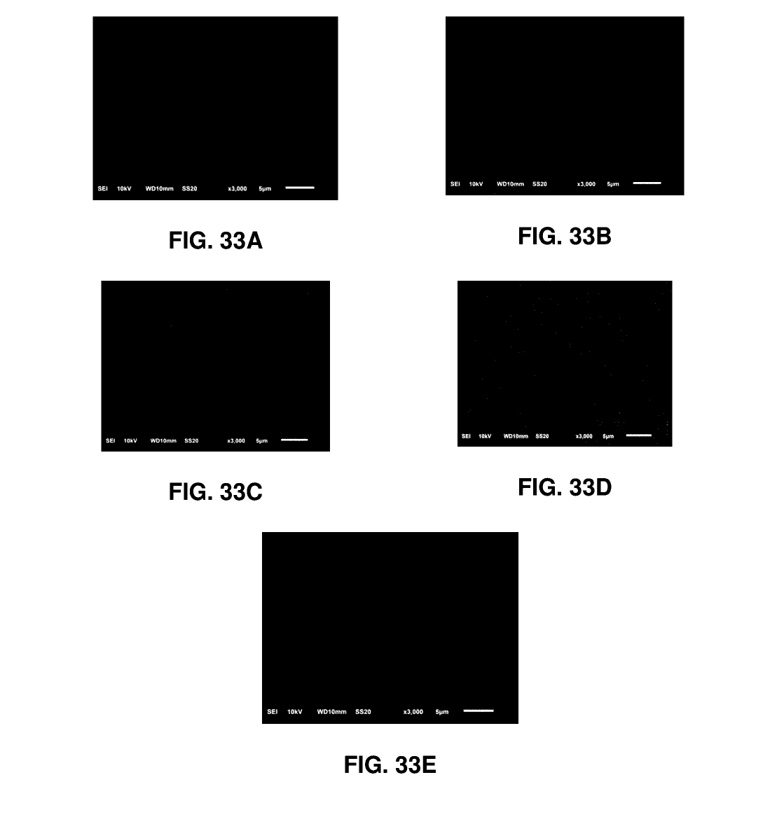

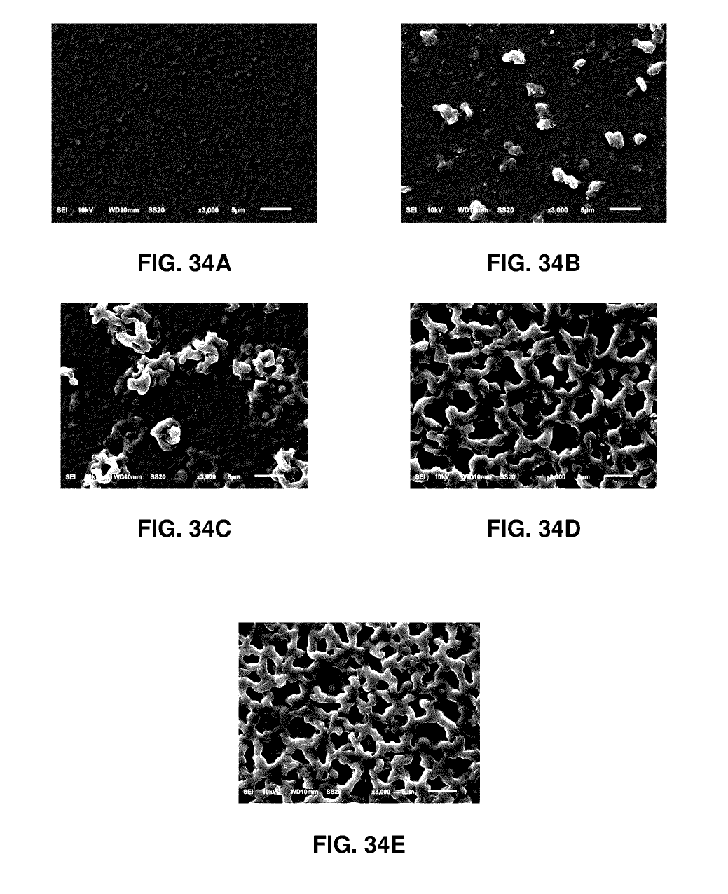

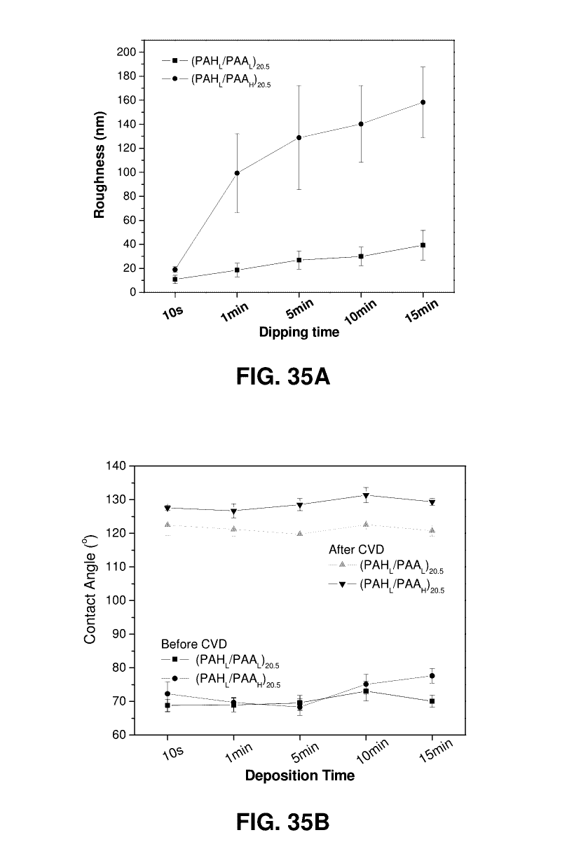

LbL assembly is able to generate smooth surface with roughness in nanometer level. Rough surface is always hard to achieve via LbL assembly of polyelectrolytes. Special LbL conditions were required to achieve a rough surface. Shen et al.[6] prepared a superhydrophobic surface via fluorinating polyelectrolyte multilayer with exponential-growth behavior. It was found that the exponential growth behavior could facilitate the formation of micro/nano hierarchical structures. The resultant surfaces exhibited superhydrophobicity after the CVD of (tridecafluoroctyl)-triethoxysilane. However, in order to achieve exponential growth, the LbL assembly was carried out while two polyelectrolytes were mostly not charged, leading to a possible issue with film stability. In addition, long processing time was also required for the adsorption of polyelectrolytes. In this work, the PAH/PAA multilayers were fabricated by the alternate deposition of PAH at pH 8.5 and PAA at pH 3.5. Then, a CVD process of Trichloro(1H, 1H,2H, 2H-perfluoro-octyl)silane was done at 130° C. for 2 hours, followed by heating at 180° C. for 2 hours to remove free fluoroalkylsilane molecules. We investigated the effect of molecular weight and dipping time on the surface roughness. The surface morphologies for (PAH_{L}/PAA_{L})_{20.5} with different dipping time are shown in FIG. 33. According to the roughness data summarized in FIG. 35(a), the roughness of (PAH_{L}/PAA_{L})_{20.5} slightly increased with the dipping time but all located in the nanoscale. When high molecular weight PAA was applied, the surface roughness was significantly enhanced. As shown in FIG. 34, the surfaces were much rougher than that of (PAH_{L}/PAA_{L})_{20.5}. In addition, the surface roughness increased significantly with the dipping time, which matches the roughness data shown in FIG. 35(a). The contact angle results are presented in FIG. 35(b). After the CVD process, the contact angle increased significantly, indicating the surface was modified by fluoroalkylsilane molecules and became hydrophobic. In addition, the contact angle values of (PAH_{L}/PAA_{H})_{20.5} after the CVD were higher than that of (PAH_{L}/PAA_{L})_{20.5} process since the roughness increased with high molecular weight PAA. However, no contact angle reached over 150° via only LbL assembly.

In order to increase surface roughness and achieve superhydrophobicity, the LbL technique has to be combined with other techniques together to control surface structures. Rubner et al.[7] fabricated a porous PAH/PAA multilayer film via LbL assembly followed with a simple acidic treatment. The surface was then coated with silica nanoparticles and modified with semi-fluorinated silane via CVD process to achieved superhydrophobicity. Zhang et al.[8] prepared the polyelectrolyte multilayers covered by gold cluster via a combination of LbL technique and electrochemical deposition. A stable superhydrophobic surface was achieved after the further modification by n-dodecanethiol. However, the combination of LbL assembly with other techniques increased the complexity of fabrication by requiring more materials and equipments and increasing the processing time. Therefore, in this work, only porous PAA/PAH multilayers were applied to achieve superwettability. The porous induction was carried out after the LbL assembly of PAA (pH 3.5) and PAH (pH 8.5). The assembled polyelectrolyte multilayer films were immersed in the water solution at a certain pH for 5 min followed by washing with DI water (pH=5.5) for 5 min. After the porous induction, the films were dried and then heated at 180° C. for 2 hours to cross-link the films and prevent the porous structures from being distorted. In the end, the same CVD process of Trichloro(1H, 1H,2H, 2H-perfluoro-octyl)silane was applied to make the surface hydrophobic.

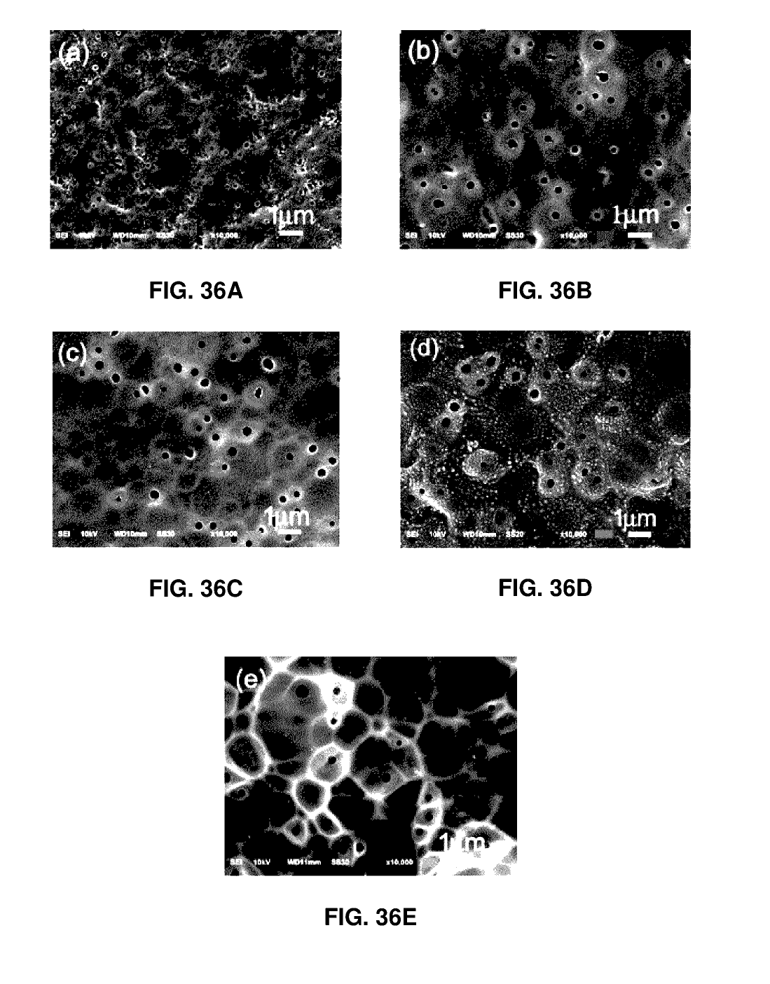

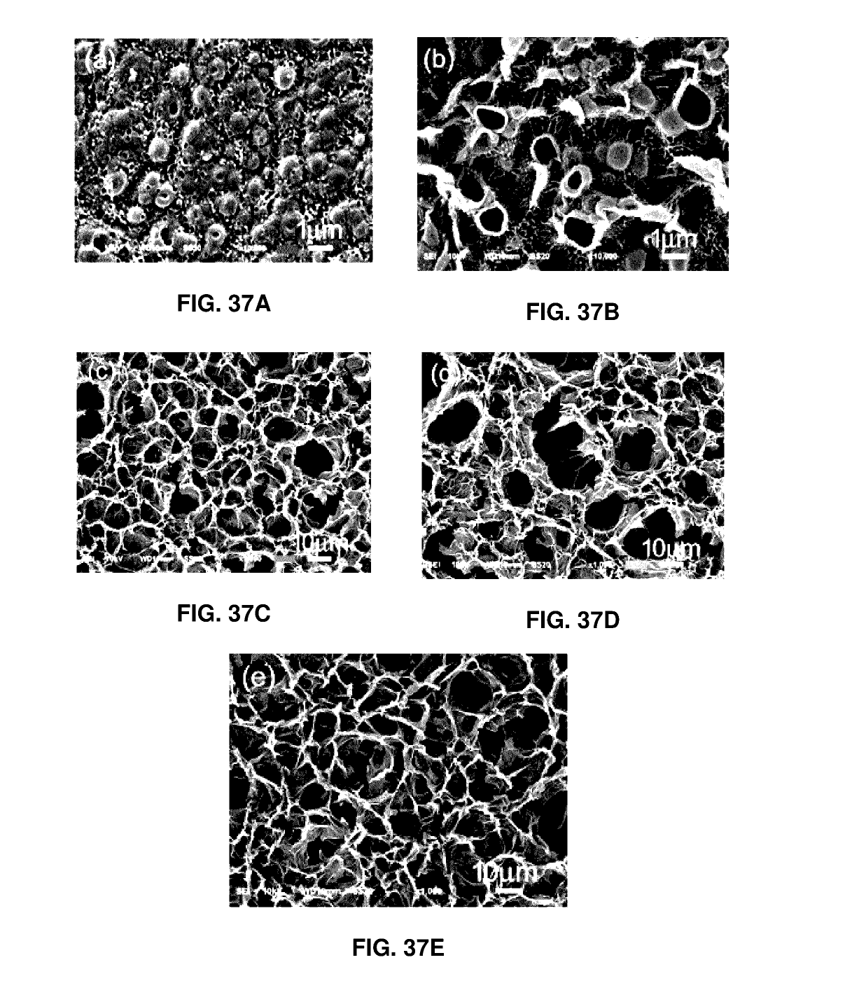

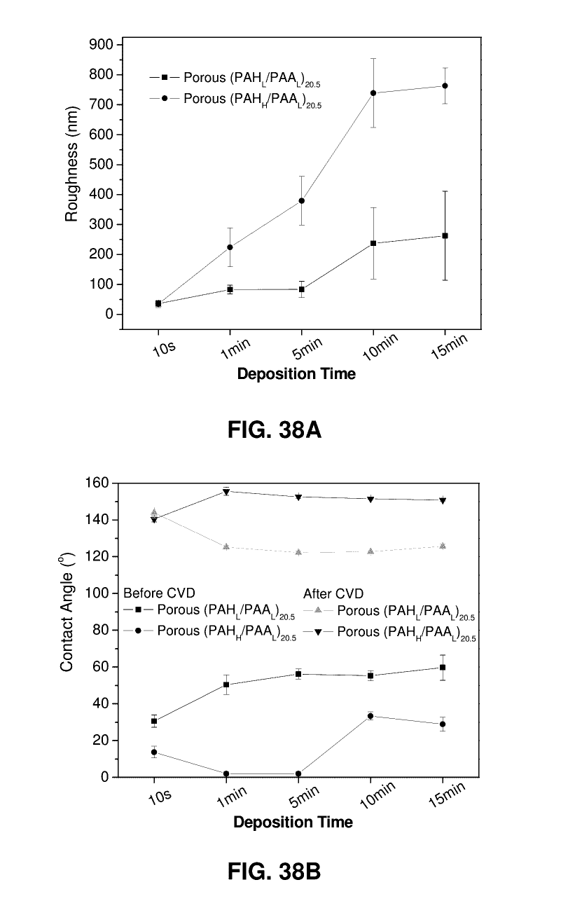

FIG. 36 and FIG. 37 include the SEM images of porous surfaces for (PAH_{L}/PAA_{L})_{20.5} and (PAH_{H}/PAA_{L})_{20.5}, respectively. For both systems, the porous induction was done at pH of 2.0. In general, the surface roughness was significantly enhanced after the porous induction, and the surface roughness increases with the increase of dipping time. For the PAA_{L}/PAH_{L} system, the porous surface was much flatter than that of (PAH_{H}/PAA_{L})_{20.5}, leading to higher contact angle before CVD process and smaller contact angle after CVD in FIG. 38(b). The roughness values summarized in FIG. 38(a) are in accord with the SEM images. According to our previous studies[9], high molecular weight PAH could provide stronger charge repulsion during the porous induction, leading to more drastic chain rearrangement and rougher surfaces. After porous induction, the surface topography became relatively complicated, with both nano and microscale structures involved. Moreover, the profiler used to measure roughness is not accurate enough for nanoscale roughness, while nanoscale roughness also affects the contact angle significantly. These explain why it is hard to build a connection between the contact angle and roughness. For PAA_{L}/PAH_{H} system, it was found that the contact angle reached over 150° when dipping time increased to 1 min. With further increase of the dipping time, the contact angle slightly decreased but still over 150°. From FIG. 37(b), it is obvious that the porous induction not only enhanced the surface roughness but also facilitated the formation of hierarchical surface topography, which leads to a successful transformation from superhydrophilicity (contact angle ˜0°) to superhydrophobicity (contact angle=155.6±2.2°) through CVD of fluoroalkylsilane molecules as presented in FIG. 38(b). Moreover, we were able to shorten the dipping time to 1 min.

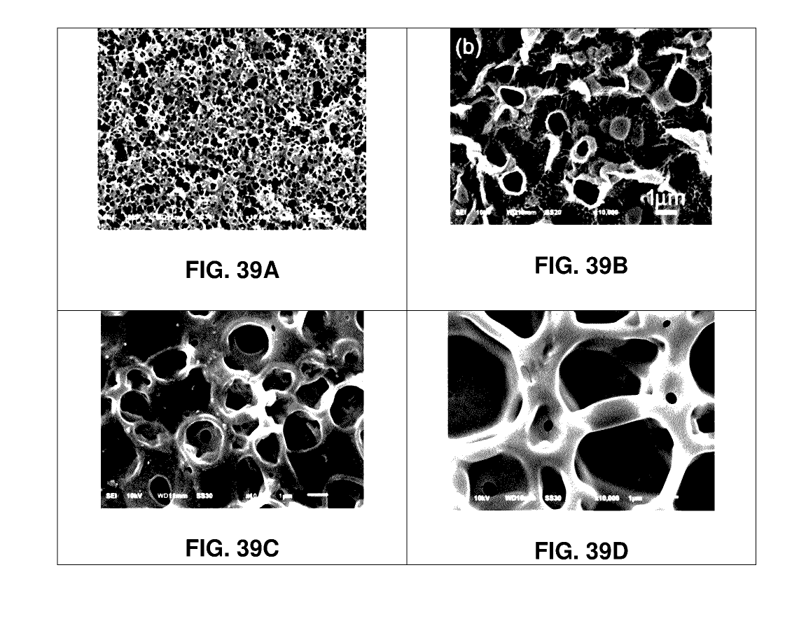

Another parameter which affects the surface topography significantly is the pH for porous induction. The (PAH_{H}/PAA_{L})_{20.5} samples with 1 min dipping were treated at pH from 1.8 to 2.4. The surface SEM images are shown in FIG. 39. The values of contact angle and roughness are listed in Table 1. It is obvious that the surface pore size increased with the increase of pH, which is consistent with the previous studies[10-12]. Lower pH facilitates the formation of nanoscale structure on the surface. The nanoscale structure disappeared when pH increased to 2.2, leading to a decrease of contact angle. It was found that when pH=2.0, the contact angle after CVD reached over 150° due to the hierarchical surface topography.

Further, we put a droplet of water-soluble ink solution on the surfaces of the (PAH_{H}/PAA_{L})_{20.5} samples with 1 min dipping and pH=2.0 for porous induction before and after CVD process, respectively. The image is shown in FIG. 32. Before the CVD process, the surface was superhydrophilic, leading to a complete wetting by the ink droplet. After the CVD process, the droplet beaded up on the surface, which means the surface turned into superhydrophobicity. The XPS spectra show that the fluoroalkylsilane molecules successfully reacted with free amine groups and were grafted onto the surface due to the appearance of a strong fluorine peak at 688 eV after the CVD process. In the XPS carbon 1 s spectrum, a peak at 294 eV corresponds to —CF₃, while a peak at 292 eV corresponds to —CF₂—. The ratio of the two peaks is around 1/5, which is consistent with the chemical structure of fluoroalkylsilane. This further confirms that the fluoroalkylsilane molecules did not go through any decomposition during the CVD process.

TABLE 1
The effect of pH for porous induction on the contact angle and roughness
of porous (PAH_{H}/PAA_{L})_{20.5} surfaces with the dipping time of 1 min

| | | Contact Angle after | Roughness |
|---|---|---|---|
| Sample | pH | CVD | (nm) |
| (PAH_{H}/PAA_{L})_{20.5} | 1.8 | 124.8 ± 3.4 | 54 ± 12 |
| | 2.0 | 155.6 ± 2.2 | 270 ± 24 |
| | 2.2 | 142.6 ± 0.5 | 137 ± 27 |
| | 2.4 | 138.3 ± 1.6 | 89 ± 8 |

In summary, by tuning the dipping time, molecular weight of polyelectrolytes and pH of the porous induction, we successfully fabricated a surface with hierarchical structure by depositing porous (PAH_{H}/PAA_{L})_{20.5}. A switch from superhydrophilicity to superhydrophobicity was achieved via a simple chemical vapor deposition of fluoroalkylsilane molecules. We were able to shorten the dipping time from conventional 15 or 20 min to only 1 min.

### REFERENCES

1. Genzer, J.; Efimenko, K., Recent developments in superhydrophobic surfaces and their relevance to marine fouling: a review. Biofouling 2006, 22 (5), 339-360.
2. Feng, L.; Zhang, Z.; Mai, Z.; Ma, Y.; Liu, B.; Jiang, L.; Zhu, D., A super-hydrophobic and super-oleophilic coating mesh film for the separation of oil and water. Angewandte Chemie International Edition 2004, 43 (15), 2012-2014.
3. Chen, Y.; Chen, S.; Yu, F.; Sun, W.; Zhu, H.; Yin, Y., Fabrication and anti-corrosion property of superhydrophobic hybrid film on copper surface and its formation mechanism. Surface and Interface Analysis 2009, 41 (11), 872-877.
4. Lai, Y.; Tang, Y.; Gong, J.; Gong, D.; Chi, L.; Lin, C.; Chen, Z., Transparent superhydrophobic/superhydrophilic TiO 2-based coatings for self-cleaning and anti-fogging. Journal of Materials Chemistry 2012, 22 (15), 7420-7426.
5. Zheng, Y.; Bal, H.; Huang, Z.; Tian, X.; Nie, F.-Q.; Zhao, Y.; Zhai, J.; Jiang, L., Directional water collection on wetted spider silk. Nature 2010, 463 (7281), 640-643.
6. Fu, J.; Ji, J.; Shen, L.; Miller, A.; Rosenhahn, A.; Shen, J.; Grunze, M., pH-amplified exponential growth multilayers: A facile method to develop hierarchical micro- and nanostructured surfaces. Langmuir 2008, 25 (2), 672-675.
7. Zhai, L.; Cebeci, F. C.; Cohen, R. E.; Rubner, M. F., Stable superhydrophobic coatings from polyelectrolyte multilayers. Nano letters 2004, 4 (7), 1349-1353.
8. Zhang, X.; Shi, F.; Yu, X.; Liu, H.; Fu, Y.; Wang, Z.; Jiang, L.; Li, X., Polyelectrolyte multilayer as matrix for electrochemical deposition of gold clusters: toward super-hydrophobic surface. Journal of the American Chemical Society 2004, 126 (10), 3064-3065.
9. Yu, J.; Sanyal, O.; Izbicki, A. P.; Lee, I., Development of Layered Multiscale Porous Thin Films by Tuning Deposition Time and Molecular Weight of Polyelectrolytes. Macromolecular rapid communications 2015, 36 (18), 1669-1674.
10. Berg, M. C.; Zhai, L.; Cohen, R. E.; Rubner, M. F., Controlled drug release from porous polyelectrolyte multilayers. Biomacromolecules 2006, 7 (1), 357-364.
11. Hiller, J. A.; Mendelsohn, J. D.; Rubner, M. F., Reversibly erasable nanoporous anti-reflection coatings from polyelectrolyte multilayers. Nature materials 2002, 1 (1), 59-63.
12. Mendelsohn, J.; Barrett, C. J.; Chan, V.; Pal, A.; Mayes, A.; Rubner, M., Fabrication of microporous thin films from polyelectrolyte multilayers. Langmuir 2000, 16 (11), 5017-5023.

We claim:

1. A polyelectrolyte multilayer thin film having pores in the film and fluorine on the surface of the film, wherein at least some of the pores have a diameter of 20 to 50 nm.

2. The film of claim 1, wherein the film comprises alternating layers of polycation and polyacrylic acid.

3. The film of claim 2, wherein the polyacrylic acid has a weight average molecular weight over 100,000 g/mole.

4. The film of claim 2, wherein the polyacrylic acid has a weight average molecular weight of about 225,000 g/mole.

5. The film of claim 2, wherein the polycation comprises poly(allyamine hydrochloride).

6. The film of claim 1, wherein the film has a thickness of 250-500 nm.

7. The film of claim 1, comprising high molecular weight polyanion.

8. A polyelectrolyte multilayer thin film, having a hydrophobic surface characterized by a contact angle with water of greater than 150°.

9. The film of claim 8, wherein the contact angle is 160° or greater.

10. The film of claim 8, wherein the film comprises alternating layers of polycation and of polyacrylic acid.

11. The film of claim 10, wherein the polycation comprises polyethleneimine, poly(allylamine hydrochloride), or DADMAC.

12. The film of claim 10 wherein the polyacrylic acid has a weight average molecular weight greater than 100,000.

13. The film of claim 10 wherein the polyacrylic acid has a weight average molecular weight of about 225,000 g/mole.

14. The film of claim 8, comprising high molecular weight polyanion.

15. A polyelectrolyte multilayer thin film comprising built up alternating layers of polycation and polyanion, wherein the polyanion comprises poly acrylic acid having a weight average molecular weight of greater than 100,000 g/mole, wherein the film is covered or partially covered with fluorine detectable by x-ray photoelectron spectroscopy.

16. The polyelectrolyte multilayer thin film according to claim 15, wherein the polyacrylic acid has a weight average molecular weight of about 225,000 g/mole.

17. The polyelectrolyte multilayer thin film according to claim 16, wherein the film has a smooth morphology.

18. The polyelectrolyte multilayer thin film according to claim 16, wherein the film has a microporous morphology.

19. The film of claim 15, having nanopores with a diameter in the range of 20 to 50 nm.

20. The film of claim 15, comprising high molecular weight polyanion.

21. A composite comprising a first PEM thin film having a microsized porous structure disposed on a second PEM thin film having a nanosized porous structure, wherein a surface of the composite comprises fluorine detectable by x-ray photoelectron spectroscopy.

22. The composite of claim 21, wherein one or both of the first and second PEM thin films comprise polyacrylic acid.

23. A membrane comprising a composite according to claim 21 disposed on a porous substrate.

24. The membrane of claim 23, wherein the porous substrate in a non-woven web.

25. The composite of claim 21, comprising polyacrylic acid having a weight average molecular weight of greater than 100,000 g/mole.
